(19) United States
(12) Patent Application Publication
(10) Pub. No.: US 2014/0194749 A1
(43) Pub. Date: Jul. 10, 2014

(54) Method and System for Detection of Cancer

(71) Applicant: Bar-Ilan University, Ramat Gan (IL)

(72) Inventors: Dror FIXLER, Ganey Tikva (IL); Rinat ANKRI, Netanya (IL)

(21) Appl. No.: 14/149,925

(22) Filed: Jan. 8, 2014

Related U.S. Application Data
(60) Provisional application No. 61/749,939, filed on Jan. 8, 2013.

(51) Int. Cl. A61B 5/00

(52) U.S. Cl. CPC A61B 5/0059; B82Y 5/00
USPC 600/473; 977/904

(57) ABSTRACT

A non-invasive and real-time optical method for detection of cancerous cells that includes the steps of optically irradiating an area of a tissue in which targeted nanoparticles are accumulated with a light source outputting an optical signal of ne or more specific wavelengths; identifying cancerous cells by measuring diffusion reflection of the irradiated tissue where the cancerous cells and the nanoparticles are located; and outputting data indicative of the identified cancerous cells.

### CROSS-REFERENCE TO RELATED PATENT APPLICATIONS

This application claims priority from U.S. provisional patent application No. 61/749,939 filed on Jan. 8, 2013, which is incorporated herein by reference in its entirety.

### FIELD OF THE INVENTION

The present invention relates to methods and systems for cancer detection and, more particularly, to such methods and systems in which optical properties of nanoparticles are measured.

### BACKGROUND OF THE INVENTION

Nanoparticle-based contrast agents for molecular imaging became a mainstay imaging tool for selectively detecting and imaging biological processes and diseases. The use of the enhanced scattering properties of gold nanoparticles as near infrared (NIR) contrast agents is under intensive investigation. This promising field builds on the safety of nonionizing radiation, ease of generation, relatively high tissue penetration depth, and reduced auto-fluorescence of the tissue in this spectral range. In addition, the particles' superior absorption properties have been utilized for photothermal therapy.

The Diffusion Reflection (DR) based medical imaging method is very attractive since it is non-ionizing, low cost, convenient to generate and detect, and highly sensitive to the optical properties of the tissue. In the last decade, several diagnostic methods were developed based on DR measurements. For example, Yang et al., 2001 suggested UV reflectance spectroscopy for DNA and protein changes probing in human breast tissues. Zhu et al., 2006 presented diagnosis of breast cancer using DR spectroscopy, where a physical model (Monte Carlo inverse model) and an empirical model (partial least squares analysis) based approaches were compared for extracting diagnostic features from the diffuse reflectance spectra. Cerussi et al., 2011 presented diffuse optical spectroscopic imaging (DOSI) as a noninvasive and quantitative method which enables the measurement of tissue hemoglobin, water and lipid. Still, as many other spectroscopic methods, the DR technique suffers from multiple scattering which dominates light propagation in tissue. Therefore, a diagnostic tool which can diminish the scattering interruption on the DR signal is desired.

### SUMMARY OF THE INVENTION

According to some aspects of the invention, there is provided a non-invasive and real-time optical method for detection of cancerous cells that includes the steps of: (a) optically irradiating an area of a tissue in which targeted nanoparticles are accumulated with a light source outputting an optical signal of one or more specific wavelengths; (b) identifying cancerous cells by measuring diffusion reflection of the irradiated tissue where the cancerous cells and the nanoparticles are located; and (c) outputting data indicative of the identified cancerous cells.

In some embodiments, the identifying step includes: (i) detecting diffusion reflection intensities of an area of the irradiated tissue for different distances between said light source and a detector; and (ii) calculating optical properties of the irradiated tissue based on the detected reflected intensity behavior in relation to the distances using a diffusion reflection based mathematical model.

The optical properties may optionally include absorption and/or scattering properties of the irradiated tissue.

In some embodiments, the irradiation is carried out with a laser device alone. In some other embodiments, the irradiation is carried out with a laser device together with at least one optical fiber for guiding light outputted from the laser device to the cancerous cells area.

In some embodiments, the one or more wavelengths used for irradiating the tissue are in the range of 650-900 nm.

According to some embodiments, the cancerous cells are of a superficial tumor, such as a head and neck cancer or melanoma.

According to some embodiments of the invention, the nanoparticles are gold nanorods (GNR). Optionally, the gold nanorods are conjugated with targeting moieties specific to receptors of the cancerous cells.

According to some embodiments, the targeting moieties are antibodies and the gold nanorods are polyethyleneglycol (PEG)-coated.

Optionally, the conjugated gold nanorods are administered to a patient before the optical irradiation of the cancerous tissue.

According to some embodiments, in which diffusion reflection models are used to identify cancerous cells in the tissue, the method further includes detecting wavelengths of light irradiated from the tissue additionally to detecting the light intensity, and identifying concentration of cancerous cells in the irradiated tissue based on red-shift of the irradiated light caused by surface plasmon resonance of concentrated nanoparticles. Optionally, in these embodiments, the tissue may be irradiated by outputting an optical signal of multiple wavelengths for enhancing identification of cancerous cells concentration.

According to other aspect of the invention, there is provided a system for non-invasive and real time optical detection of cancerous cells in which targeted nanoparticles are accumulated, where the system includes: (a) an optical source setup for irradiating the tissue, where the optical source includes a laser device configured for outputting an optical signal of at least one wavelength; (b) at least one detector configured for detecting light reflected from the irradiated tissue; and (c) a processing unit for receiving output of the at least one detector and identifying cancerous tissue by calculating optical properties of the irradiated tissue from the detected light, using a diffusion reflection based mathematical model.

The optical source setup may optionally further include one or more optical fibers for guiding light outputted by the laser device to the cancerous cells area. In some embodiments, the laser device is configured for outputting an optical signal of a single wavelength or multiple wavelengths.

According to some embodiments, the optical source setup further includes at least one micrometer plate attached to a distal edge of said at least one optical fiber for allowing changing the relative source-detector separation between the location of the optical fiber output and the at least one detector for measuring the diffusion reflection in the specific body area.

Additionally or alternatively, the system further includes a signal collecting unit for collecting output signals from the at least one detector and outputting signal related data, where the signal collecting unit is configured to transmit the signal related data to the processing system.

The signal collecting unit may be, for example, an oscilloscope, a central processing unit (CPU) communicating with said processing unit or a software program operable through the processing unit capable of receiving input data from the at least one detector through hardware of the processing unit.

Optionally, the at least one detector includes at least one photodiode.

According to some embodiments, the optical source setup and/or the at least one detector are/is configured for changing their/its location for measuring irradiated light from various source-detector separations.

The detector may be optionally configured for being moved at predefined distance intervals for changing its relative location and/or the optical source setup is configured for being moved at predefined intervals for changing the relative location of an output of the light source.

According to some embodiments, the detector and/or the optical source setup are/is configured to allow continuous measuring of spatial reflectance from the irradiating tissue.

The optical source setup may optionally include at least one laser diode, each outputting an optical signal at a different narrow wavelength.

According to some embodiments, the at least one detector is further configured for detecting wavelength or frequency of the optical signal irradiated from the tissue and the processing unit is configured for identifying concentration of cancerous cells in the irradiated tissue based on intensity decay of the optical signal, caused by concentrated targeted nanoparticles. The laser device may optionally be configured for outputting an optical signal of multiple wavelengths, wherein the red-shift is measured for various wavelengths.

According to some embodiments, the optical properties measured by the one or more detectors include absorption and/or scattering properties of the irradiated tissue.

According to some embodiments of the invention, there is provided a non-invasive and real-time optical method for detection of cancerous cells, wherein the method includes the steps of: (a) optically irradiating with a light source outputting an optical signal of at least one wavelength, an area of a tissue in which targeted nanoparticles are accumulated; (b) identifying cancerous cells by measuring at least one optical property of the diffuse light from the irradiated tissue where the cancerous cells and the nanoparticles are located; and (c) outputting data indicative of the identified cancerous cells.

According to some embodiments, the optically irradiating of the tissue area is carried out by using a light source configured for outputting a multi-wavelength, high bandwidth optical signal, wherein the identification of cancerous cells is carried out by using a frequency domain photon migration (FDPM)-based model.

### BRIEF DESCRIPTION OF THE DRAWINGS

FIG. 1 is a flowchart illustrating a method for detection of cancerous cells, according to some embodiments of the invention.

FIG. 2A schematically illustrates a system for detection of cancerous cells, according to some embodiments of the invention.

FIG. 2B schematically illustrates a system for detection of cancerous cells that includes a laser diode source for emitting NIR light, according to some embodiments of the invention.

FIG. 3 is a flowchart illustrating a method for detection of cancerous cells using detector/source displacement, according to some embodiments of the invention.

FIG. 4 is a diagram showing the absorption spectra of gold nanorods (GNR).

FIG. 5 is a diagram showing the logarithm of the detected intensity as a function of the source-detector separation (the distance between the output of the laser's optical fiber source and the photodiode detector) for five phantoms that differ by their absorption coefficients, according to some implementations of the invention.

FIG. 6 is a diagram showing the resulting logarithm of the detector intensity as a function of the source-detector separation for one homogenous phantom and for two phantoms with different concentrations of GNR.

FIG. 7 shows the slopes of the intensity logarithm for cancerous and normal cells measured before administration of GNR into mice, a short while after their administration (t=0) and more than ten hours post administration (t>10 h).

FIG. 8 shows the measured diffusion reflection intensity in a semi-logarithmic scale as a function of source-detector separation for various tissue types (tumorous and non-tumorous) before injection of nanoparticles and ten hours or more after their injection.

FIG. 9 shows UV-Vis absorption spectra (normalized) of 3% India Ink (dotted line); bare GNR₆₅₀ (25×65 nm, thin dashed line); PEG coated GNR₆₅₀ (thick dashed line); anti-EGFR coated GNR₆₅₀ (dotted-dashed line) and bare GNR₇₈₀ (52×13 nm) (solid line).

FIGS. 10A-10B show measured diffusion reflection intensity in semi-logarithmic scale as a function of the source-detector separation. 10A: for different phantoms used in a second line of experiments: a homogeneous phantom with a reduced scattering property of μ′ₛ˜1.45 mm⁻¹, and an absorption coefficient of μₐ=0.0137 mm⁻¹ following 650 and 780 nm illuminations (the triangle marked line and the solid line, respectively) and a solid phantom containing 0.01 mg/ml GNR₆₅₀ following 650 and 780 nm illuminations (the crossed line and circle marked line, respectively); 10B: for the same homogeneous phantom following 650 nm and 780 nm illumination (the triangle marked line and the solid line, respectively) and solid phantom containing 0.02 mg/ml GNR₇₈₀ following 650 and 780 nm illuminations (the cross line and circle marked line, respectively).

FIG. 11 shows a comparison between the Δ slopes (absolute values) of the reflected light intensity from phantoms containing GNR₆₅₀ following 650 and 780 nm illuminations at different GNR concentrations.

FIG. 12 shows the measured absorption spectra (normalized absorption vs. wavelength) of GNR₆₅₀ from two slides presenting densities of 0.0155 mg/cm² (dashed line) and 0.0372 mg/cm² (dashed-dotted line).

FIGS. 13A-13B show diffusion reflection intensities (in a semi-logarithmic scale) ln(Γ(ρ)) as a function of the source-detector separation ρ. FIG. 13A presents the DR profiles of cancerous tissue with a relatively low GNR₆₅₀ concentration; while the reflectance slope following 780 nm illumination presents the same value as before illumination (circle and asterisk marked lines before and after illumination, respectively), 650 nm illumination introduced a sharper slope (triangle marked line) compared to the slope before the GNR injection (solid line). The graph in FIG. 13B indicates the DR profiles of tested cancerous and non-cancerous tissues presenting a higher GNR₆₅₀ concentration. The DR profiles of the tumor following 650 nm and 780 nm illuminations (triangle and asterisk marked lines, respectively) introduced an increase in the curves' slopes compared to the non-cancerous tissue before the GNR₆₅₀ injection (solid and circle marked lines).

FIG. 14 shows the DR intensity profile results from Monte Carlo simulation: ρ²Γ in semi-logarithmic scale as a function of source-detector separation ρ: ln(ρ²Γ(ρ)), for a simulated homogeneous tissue presenting four different absorption coefficients μₐ=0.0115 mm⁻¹ (diamond marked line); 0.0126 mm⁻¹ (circle marked line); 0.018 mm⁻¹ (triangle marked line); and 0.0227 mm⁻¹ (square marked line).

FIG. 15 shows the theoretically calculated and simulated curves indicating the linear dependence of the square slopes of the profile ln(ρ²Γ(ρ)) for the same simulated tissues mentioned in FIG. 14.

FIG. 16 shows DR profile, ln(ρ²Γ(ρ)), of four tissue-like phantoms having a constant intralipid (IL) concentration, resulting in a constant reduced scattering coefficient, and different ink concentrations resulting in different absorption coefficients of: μₐ=0.0115 mm⁻¹ (solid line); 0.0126 mm⁻¹ (marked as “x”); 0.018 mm⁻¹ (marked as “*”); and 0.0227 mm⁻¹ (marked as “°”).

FIG. 17 shows the linear dependence of the ln(ρ²Γ(ρ))square slopes of solid phantom with different absorption coefficients on the absorption coefficients.

FIG. 18 shows spectrometric results for GNR absorption vs. GNR concentration.

FIG. 19 shows experimental results of the DR measurements for three phantoms containing different concentrations of GNR: 0.0022 mg/ml (solid line); 0.003 mg/ml (marked by “x”); and 0.0057 mg/ml (marked by “*”).

FIG. 20 shows in vivo results of tumor bearing mice, showing the DR profile, plotted in the logarithmic form ln(ρ²Γ(ρ)), as a function of the source-detector separation ρ: before GNR injection (solid line); 15 min post GNR injection (asterisk marked line); 5 hours post GNR injection (cross marked line); and 10 hours post GNR injection (circle marked line).

### DETAILED DESCRIPTION OF SOME EMBODIMENTS OF THE INVENTION

In the following detailed description of various embodiments, reference is made to the accompanying drawings that form a part thereof, and in which are shown by way of illustration specific embodiments in which the invention may be practiced. It is understood that other embodiments may be utilized and structural changes may be made without departing from the scope of the present invention.

The present invention, in some embodiments, provides a method for detection of cancerous cells based on measurement of optical properties of targeted nanoparticles accumulated in a tissue. The method is noninvasive, non-ionizing optical detection method that provides a highly sensitive, simple and real time tool for cancer detection.

The nanoparticles for use in the present invention may be selected from noble metal nanoparticles, e.g., gold, copper, silver, or a combination of them, that present highly tunable optical properties, which can be easily tuned to desirable wavelengths according to their shape (e.g., nanoparticles, nanoshells, nanorods, etc.), size (e.g., 1 to 100 nm), and composition (e.g., core/shell or alloy noble metals), enabling their imaging applications under native tissue.

These nanoparticles can also be easily functionalized with/conjugated to various moieties, such as antibodies, peptides, and/or DNA/RNA to specifically target different cells, or with a biocompatible polymer, e.g., polyethylene glycol (PEG).

The term ‘targeted nanoparticles” as used herein refers to nanoparticles as described above which are configured in a way such that they bind specifically to cancer cells and thus accumulate in a cancerous tissue.

In certain embodiments, the nanoparticles used in the present invention are gold nanoparticles such as gold nanorods (GNRs). Since most cancer cells present epidermal growth factor receptor (EGFR) molecules on their surface, in certain embodiments the gold nanoparticles can be conjugated to an anti-EFGR antibody, e.g., Cetuximab, forming targeted nanoparticles that home specifically to cancerous cells. In some embodiments, the cancerous cells may be, without being limited to, squamous cell carcinoma of head and neck cancer or melanoma.

The targeted nanoparticles can be administered to an individual by any suitable mode of administration, for example, by intravenous injection. Once administered, the targeted nanoparticles will accumulate in cancerous cells, if present in said individual.

The distance between the detector(s) detecting the irradiated light from the tissue and the light source emitting light for irradiating the same tissue is defined herein as “source-detector separation”.

According to certain embodiments of the method of the present invention, an area of a tissue in which the targeted nanoparticles are accumulated is optically irradiated with a light source that outputs optical signal of one or more wavelengths. Cancerous cells in this tissue can be identified by detecting intensity of light emitted from the irradiated tissue for different distances between the light source and the detector(s) and calculating optical properties such as absorption and/or scattering coefficients of the irradiated tissue based on diffusion reflection mathematical models which define the relation between the intensity, the source-detector separation and the absorption/scattering coefficients of the tissue.

The experimental and theoretical aspects and details of the present invention have been disclosed by the inventors in the following publications: Ankri R et al, 2012 (a), Ankri R et al. (2012b), and Ankri R et al. (2013), all of which are incorporated by reference herein in their entirety as if wholly described therein.

The diffusion reflectance (DR) profile of an irradiated tissue depends on its absorption and scattering coefficients (Jacques et al., 2008). The absorption coefficient of a tissue is predominantly determined by the concentration of the absorbance molecules, while the scattering coefficient depends mainly on the size and shape of the scattering components in the tissue, rather than their concentration (Shimada et al., 2009). Since imaging techniques that are based on scattering (with or without nanoparticles as contrast agents) suffer from relatively high background noise and low contrast, the diffusion reflection (DR) method is designed herein to focus mainly on the absorption properties of the targeted nanoparticles rather than their scattering properties. As a result, no contrast interruptions are expected.

Since GNRs have unique size- and shape-dependent optical properties, they can cause a significant change in the optical properties of the targeted tissue. Previous studies have presented diffuse reflectance measurements for cancer diagnosis (Bigio et al., 2000) but without nanoparticles as contrast agents. The diffusion reflectance method described in the present invention presents higher efficiency and sensitivity resulting from the GNR insertion that specifically target cancerous cells and significantly change their absorption.

In certain embodiments, the invention relates to measuring the diffusion reflectance for head and neck cancer (HNC) using GNRs that are targeted to HNC tissues. The head and neck lymph nodes are located adjacent to the skin where visible-NIR light can easily penetrate when using a light source that radiates the tissue in a noninvasive manner by, for example, placing an outlet of an optical fiber connected to a laser light source over the patient's skin in the head and/or neck area where a cancerous tumor is suspected to be located, after targeted nanoparticles have been administered.

One of the major diagnosis challenges in HNC today is reliable detection of involved lymph nodes, since their status is one of the most important prognosis predictors and is also pivotal for appropriate treatment. However, assessment of lymph nodes based on currently available imaging techniques is limited in sensitivity and specificity and fails to distinguish between non-neoplastic and malignant processes. These limitations lead to the routine performance of prophylactic procedures such as extensive neck dissection and radiation. Hence, the development of more sensitive in vivo detection techniques is of major importance and could substantially improve HNC treatment and potential cure.

According to some embodiments, the method of the present invention is applied for HNC detection, wherein targeted EGFR-conjugated GNRs are intravenously injected into the patient, and the diffusion reflection technique is used to detect cancer based on the absorption coefficient differences between cancerous and normal tissues in a specific head and/or neck area of said patient.

The diffusion reflection is based on a diffusion model (Jacques et al., 2008), which assumes that light can be treated as a concentration of optical energy that diffuses down a concentration gradient. The loss of energy is caused by the absorbing and scattering of components within the tissue (Jacques et al., 2008). The diffusion model can solve several classes of image or property recovery problems. One of the most common among them is the measured Γ(ρ) function. This Γ(ρ) function, which describes the reflected light intensity (defined as Γ) at the tissue surface in several light source-detector separations (defined as ρ), presents a strong correlation to the tissue optical properties, such as the absorption coefficient μₐ and the reduced scattering coefficient νₛ′. The intensity of the reflected light Γ(ρ) function is described by the general formula of (Schmitt et al., 1990 and Farrell et al., 1992):

Γ(ρ)=[c₁/(ρ)ⁿ]·exp(−μρ), referred to hereinafter as Equation 1.

C₁ is a constant, depending on the optical properties of the medium and on the sizes of the source and detector apertures; n is the power of ρ, which depends on ρ's range and on the ratio μₐ/μₛ′, μ is an effective attenuation coefficient given by μ=√(3·μₐ·μₛ′), referred to hereinafter as Equation 2 (Jacques et al., 2008).

“n” is the power of ρ, which depends on ρ's range and on the scattering properties of the tissue (Farrell et al., 1992). “n” depends also on the tissue absorption properties, as in the limit of zero absorption n is nearly 2 (Schmitt et al., 1990). In the case of n=2, the reflectance profile is highly sensitive to the optical properties of the tissue and, as a result, better distinguishes between absorption coefficients that only slightly differ from each other.

By inserting n=2 to Equation (Eq. 1), it can be rewritten as:

ln(ρ²Γ(ρ))=μ*ρ, referred to hereinafter as Eq. 3.

Eq. 3 presents a linear correlation between ln(ρ²Γ(ρ)) and μ.

Resulting from Eq. 2 and Eq. 3, the square slope of the linear curve depends on the product between the absorption and the reduced scattering coefficients of the tissue.

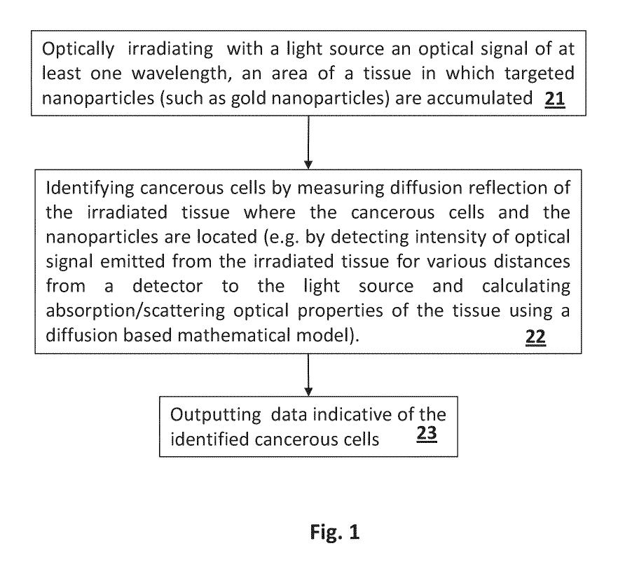

Reference is now made to FIG. 1, which is a flowchart schematically illustrating a method for detection of cancerous cells in a tissue area using targeted nanoparticles, according to some embodiments of the invention.

The method, in these embodiments, includes: (i) optically irradiating with a light source outputting an optical signal of at least one wavelength, an area of a tissue in which targeted nanoparticles (such as gold nanoparticles) are accumulated 21, using a light source within one or more specific known wavelengths suitable for excitation of the administered targeted nanoparticle; and (ii) identifying cancerous cells by measuring diffusion reflection of the irradiated tissue where the cancerous cells and the nanoparticles are located 22; and (iii) outputting data indicative of the identified optical properties of the tissue area 23.

In certain embodiments, the method is used for detection of superficial tumors such as for detection of head and neck cancer, and the light source (e.g. a laser diode or any other laser configured for outputting monochromatic optical signals in the NW range) is located in or guided to an external body area that is in proximity to the inner tissue area that is to be irradiated. The emitted light can non-invasively penetrate the skin to reach inner tissue thereof for irradiation of the internal tissue.

Cancerous cells in the tissue area can be identified, according to some embodiments, by detecting intensity of optical signal emitted from the irradiated tissue, for example using one or more optical detectors such as photodiodes adapted to detect optical signals of wavelengths/frequencies in the range of the irradiated tissue, for various distances from the detector to the light source and calculating absorption/scattering optical properties of the tissue including the accumulated nanoparticles, using a diffusion reflection based mathematical model.

The optical properties deduced from the detected intensities (e.g., deduced from amplitudes of the detected signals) and optionally also from wavelength properties in correlation to the source-detector separations (distances between the light source and the detector) may be further processed using image analysis to convert the data received from the detector(s) into a live image of the tissue area in which the areas therein identified as having cancerous cells are indicated (e.g. by a distinguishable color), allowing identification of the location of the cancerous cells in this tissue area.

According to certain embodiments, a designated computer program may be used, which enables receiving and processing the detector's output data according to predefined algorithms capable of producing, inter alia, imagery output showing the irradiated cancerous cells and for calculating the location of each irradiating spot (identified as a cancerous cells location) and its associated intensity related value. This program may also be configured for identifying borders of a tumor by identifying where concentrations of the targeted particles (coloring the overall image of the tissue) rapidly decrease, for example, or by using any other method(s) for border identification that relate to the DR imagery of the tested tissue/area.

According to certain embodiments of the invention, the nanoparticles are gold nanorods. To prevent aggregation, to stabilize the particles in physiological solution and to improve blood circulation time, the gold nanorods can be coated with a layer of polyethylene glycol (mPEG-SH, for example of molecular weight MW=5.000 g/mol). This layer also provides the chemical groups that are required for conjugation with the antibodies (SH-PEG-COOH, MW+3400 g/nol) as described according to the invention. Thus, in certain embodiments, the antibodies are conjugated to polyethyleneglycol coated-gold nanorods.

To allow the gold nanorods to bind to cancerous cells, the gold nanorods may be conjugated with targeting moieties specific to receptors present on the surface of the specific cancerous cells. For instance, to allow the gold nanorods to bind to cancerous cells of head and neck cancer, the targeting moieties are antibodies to epidermal growth factor receptor (EGFR); for binding to HER2 (human EGFR2)-positive breast cancer cells, the gold nanorods are conjugated to anti-HER2 antibodies such as Herceptin; and for binding to melanoma cells, the gold nanorods are conjugated with antibodies to fibroblast growth factor receptors (FGFR).

The targeted nanoparticles, e,g, the antibody-conjugated gold nanorods, are administered to an individual suspected of having cancer, i.e., a patient, at a certain time prior to the optical irradiation of the cancerous tissue for allowing them to accumulate in the tissue. The detection of cancerous cells in the tissue should start after a minimum accumulation period for allowing the nanoparticles to reach the designated tissue area and to bind to the cancerous cells.

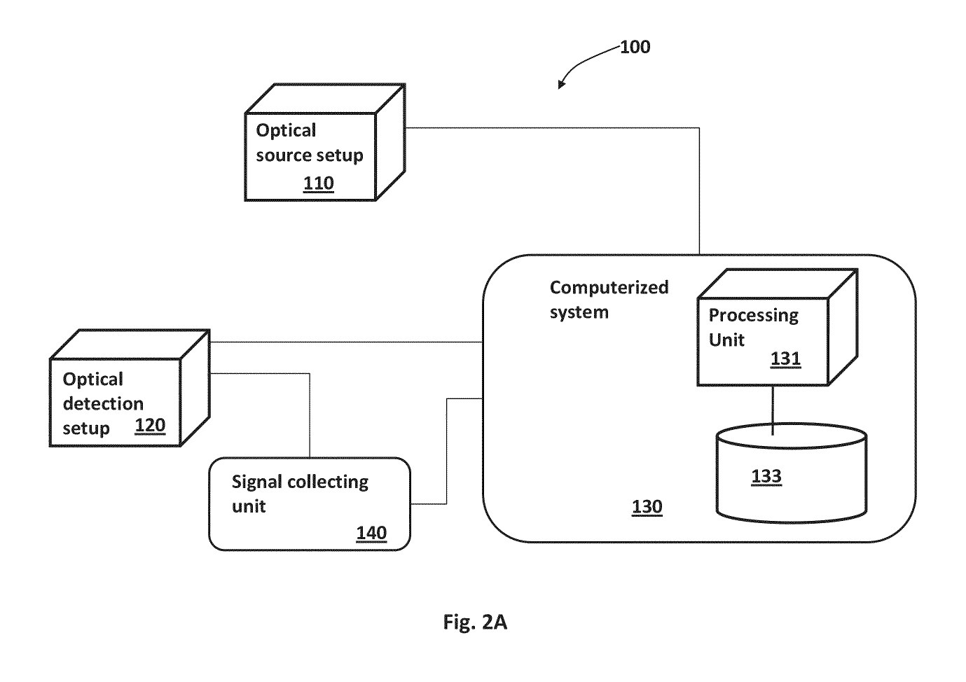

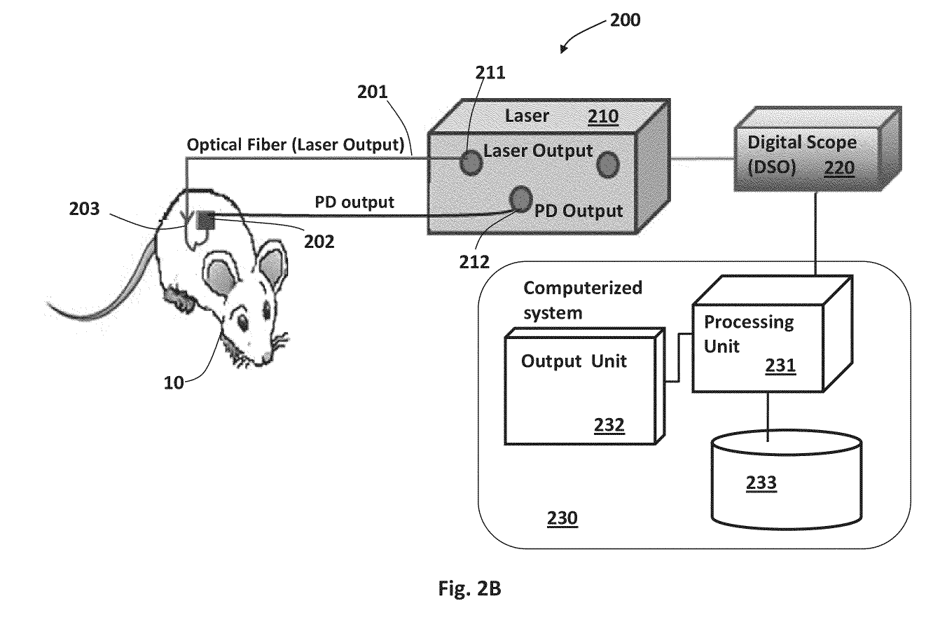

Reference is now made to FIG. 2A showing a block diagram, which generally portrays a system 100 for cancer detection, according to some embodiments of the invention. The system 100 includes an optical source setup 110 configured for outputting an optical signal (beam) at one or more wavelength or wavelength bands that correspond to excitation wavelength/wavelength band of the targeted nanoparticles (e.g. conjugated gold nanoparticles such as gold nanorods) administered to a patient and for noninvasively irradiating inner and/or external tissues of the patient's body in one or more selected body areas. This means that the output of the optical source setup 110 (e.g. a laser device that outputs a monochromatic coherent optical signal at a predefined wavelength where the output light thereof is guided via an optical fiber) is placed over or near the patient's skin to irradiate internal and/or external tissue proximal to the positioning of the optical source output. The system 100 also includes a detection setup 120 including one or more detectors such as one or more photodiodes or cameras such as IR cameras, or a charged coupled device (CCD) cameras and the like, for noninvasively detecting light reflected from the irradiated tissue. This means that each detector is placed over or in proximity to the patient's skin for detecting light scattered from the irradiated tissue including the irradiating targeted and bound nanoparticles therein. In cases in which the system 100 is used for detecting cancerous tissue, located in proximity to the skin surface, inside the patient's body, the detector and the light source are placed over or very close to the patient's skin in the area in which cancerous tissue is suspected to be found.

Output of the detector(s) from the detection setup 120 is collected and processed at a computerized system 130 having one or more processors 131 and one or more data storage unit such as a database 133.

The processing unit is configured to operate one or more software based applications/algorithms for receiving data indicative of the detected light (e.g. indicative of the amplitude/intensity detected for each source-detector separation and optionally also of the wavelength/frequency of each amplitude/intensity) and calculate according to the received data, absorption and/or scattering properties of the irradiated tissue for identifying cancerous cells therein and their location in the tissue. To identify those optical properties, the diffusion reflection methodic is used, where the relation between the intensity (which may be deduced from signal amplitude detection) and source-detector separation is taken from the above-described equations.

The system 100 optionally includes a signal collecting unit 140 in cases in which the output of the detector is not directly transmitted to the processing unit but through a mediating hardware/software means such as through an oscilloscope, a computer processing unit (CPU), for example. For example, the data from the detection setup 120 may be transmitted through cables to the computerized system 130, which may operate a designated LabView™ program configured for converting the detector data into computer readable information for identifying, for instance, the intensity vs. the source-detector separation values and optionally also the frequency/wavelength thereof.

The detector may be configured for measuring light signal intensity (amplitude) and frequency. The detected intensity is then analyzed in respect to each source-detector separation value it is associated with, to allow calculating the diffusion reflection (DR)-based absorption/scattering properties of the tissue for detection of cancerous cells therein.

The optical source setup 110 may include any known in the art light source that is configured to produce light of the desired wavelength/wavelength band such as a laser diode source, a Xenon illumination source and the like. The optical source setup 110 may also include optical devices and elements for noninvasively directing and/or guiding light to the selected external body area from which the tissue is to be irradiated such as one or more optical fibers, one or more lenses and/or phase elements, filters and the like.

According to some embodiments of the invention, the optical source and detection setups 110 and 120, respectively, are combined in a single device that is configured for both transmitting and detecting optical signals over the skin of a patient. Optionally, the combined device includes a processor for on-chip processing of the detected signals from the optical detector(s) configured for carrying out at least some of the required processing or for conversion of the signal into computer-readable data.

Reference is now made to FIG. 2B, which schematically illustrates a system 200 for tumor detection that uses a laser diode source device for emitting NIR light, according to some embodiments of the invention. This system 200 includes a laser diode based laser device 210 configured for outputting an optical signal at one or more narrow wavelength band such as at 650 nm having an optical fiber 201 connected to its output 211 for directing the outputted light therethrough to allow noninvasively irradiating the desired inner and/or outer tissue of the respective patient by approximating the output of the fiber 201 to the patient's 10 skin. The system 200 additionally includes one or more optical sensors configured for sensing light scattered from the irradiated tissue at the wavelength range of the scattered light adapted for light scattered from the particular type of targeted nanoparticles being used, such as a photodiode (PD) 202. The location of the PD can be shifted to allow changing the distance between the PD 202 and the fiber 201 output (source) for measuring intensity of reflected light from the irradiated tissue at different source-detector separations “ρ”.

The output of the optical fiber 201 may optionally be coupled to localizing device such as to a micrometer plate 203 for allowing easy relocation of the fiber 201 outputting end and holding it in each location in respect to the patient's skin, to change the distance between the light source and the detector.

According to some embodiments, the optical fiber 201 is configured for guiding optical signals (beams) at one or more wavelengths/frequencies or wavelength/frequency ranges adapted to the output of the laser diode.

According to this method, the source-detector separations “ρ” is changed over time by changing the distance between the light source (e.g. end of output of the fiber 201) and the PD 202 by changing over time the location of at least one of: the fiber 201 output end and/or the PD 202 and taking a measurement of the intensity of the irradiated tissue at this location of the PD 202 for each source-detector separations “ρ”.

According to other embodiments of the invention, the intensity is measured for various source-detector separations “ρ” simultaneously. This may be abled by having a system in which there are multiple PDs each located at a different location near the area that is to be tested for tumor detection. According to other embodiments of the invention, the optical sensor includes one or more optical cameras sensitive to the respective wavelength being used, each camera configured for simultaneously measuring the intensity of irradiated light from within the tissue for several source-detector separations.

According to some embodiments of the invention, as illustrated in FIG. 2B, the system 200 also includes a digital scope 220 such as a digital storage oscilloscope (DSO) and a computerized system 230 communicative with the digital scope 220. The scope 220 is configured for collecting the reflected intensity Γ(ρ) (in volts). The intensity measurements data is transferred whether in real time or not, to the computerized system 230 for further processing thereof. The computerized system may include, as illustrated in FIG. 2B, any known in the art computerized means for receiving, transmitting, storing, processing and outputting of data such as a processing unit 231, one or more output units such as a screen 232 and a data storage unit (e.g. database) 233.

The processing unit 231 receives the raw data from the digital scope 220 indicative of intensities of reflected light measured by the PD 202, and analyzes this data to calculate one or more related measures associated with these detected intensities in response to known source detector separation values each associated with a different intensity detection, which may be known, where the relation between the source-detector separation and the reflection intensity (or a logarithm thereof) corresponds to the diffusion reflection (energy concentration gradient) of light of the irradiated tissue.

According to other embodiments of the invention, methods and systems based on dark field microscopy and imaging may be used for detection and analysis of the reflected (scattered) light, where scattered beams are excluded from the imaging.

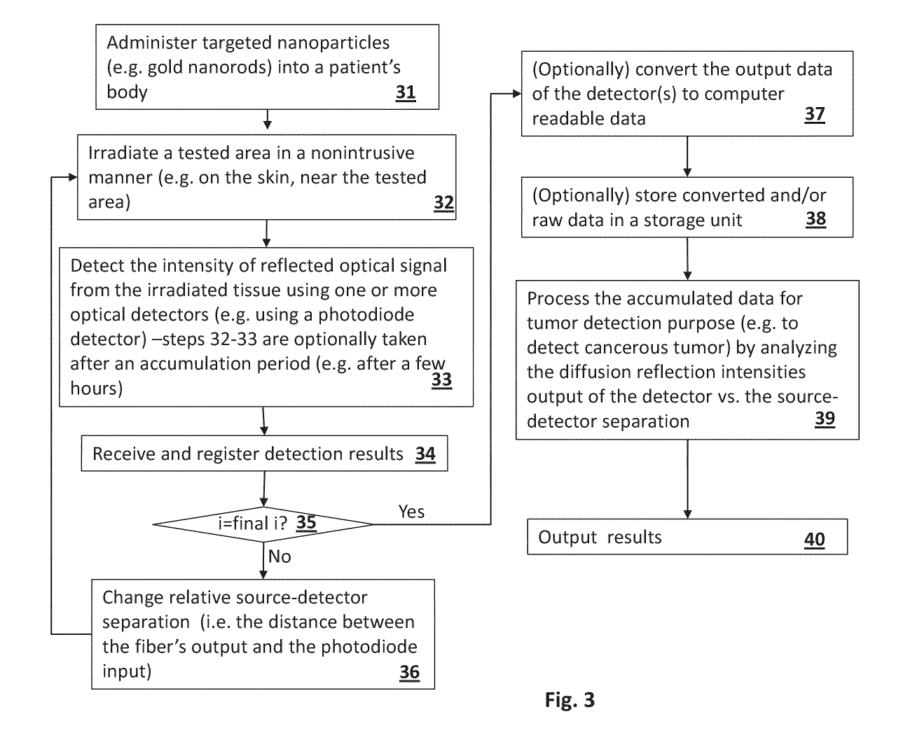

Reference is now made to FIG. 3, which is a flowchart schematically illustrating a detailed method for detection of cancerous cells, using conjugated gold nanorods (GNR), according to some embodiments of the invention. This method includes administering the GNR to the patient 31. After the administration of the targeted nanoparticles 31, the tested tissue is noninvasively irradiated 32 with an optical signal of at least one wavelength within a predefined range (such as the NIR range) and corresponding irradiated tissue detection is carried out 33.

The irradiation of the tissue is carried out, for example, by using a laser diode source and an optical fiber that guides and directs the coherent monochromatic laser light beam therethrough and outputs it in proximity or over the patient's skin in proximity externally and the tested tissue. The detector used may include one or more PDs or a CCD camera placed in proximity or over the patient's skin close to the output of the optical fiber. The source-detector separation is changed at each measurement over time 35-36 by, for example, changing a location of the optical fiber output and/or by changing the location of the PD for changing the respective distance therebetween, which defines the source-detector separation “ρ”. This allows measuring the irradiation for various values of source-detector separations for identifying the diffusive behavior of light in the tested tissue.

The detector detects the intensity/amplitude of optical signals of light reflected from the tested tissue behind the skin in the area where the cancerous cells are located, and outputs a signal/data indicative of that intensity/amplitude of the reflected light. This output data is then optionally converted to a computer/processor readable data 37 and stored 38 in a computerized storage. The accumulated data including the intensity/amplitude values of various source-detector separations is then processed 39 by a computerized system (e.g. PC computer) for analyzing DR optical properties indicative of how the irradiated light diffuses through the tested tissue to detect cancerous tissue portions/cells in the tested tissue. The results of the processing may be outputted 38 using imagery presentation of the tested tissue that shows thereof in multiples colors where each color represents the presence of the targeted GNR indicating the different concentrations thereof over the tested tissue, which may indicate the location and presence of tumorous (cancerous) cells/tissue portions for identifying borders of the tumor in the tested area.

According to some embodiments of the present invention, the method additionally includes identifying concentration of cancerous cells in the tissue by identifying concentration of nanoparticles accumulated over the cancerous cells therein by using an additional measurement and processing method that is based on intercepting surface plasmon resonance (SPR) occurring when the nanoparticles are densely accumulated in the tissue.

SPR is achieved by using light (such as infrared (IR) or NIR mono or multi chromatic laser beam) for excitation of metallic surfaces of nanoparticles causing oscillations thereof. These oscillations exhibit enhanced near-field amplitudes at the resonance wavelength, where this field is localized, meaning that the field amplitude decreases dramatically when distance from the nanoparticles surface increases, providing thereby a high spatial resolution, allowing easy distinction between the resonating nanoparticles surfaces and their non-resonating environment. This resonating causes a slight yet distinguishable red-shift in the wavelength of the light irradiated from those inter-coupled nanoparticles allowing identification thereof by detecting the wavelength/frequency of the optical signal irradiated from the tissue (in addition to detection of the signal's intensity/amplitude).

In this method, the area where the GNR are highly concentrated (the tumor area/peripheries) is distinguished from the normal concentrated healthy tissue. In the normal area the GNR spectrum is regular. The inter-particle plasmon resonance pattern of the highly concentrated GNR leads to an extension and a red-shift (42) in the absorption spectrum of the concentrated GNR and thereby allows identifying the cancerous tissue after a much lower accumulation time.

Different sizes and concentrations of the administered nanoparticles can be used to improve identification of the red-shift as well as using multi-chromatic source such as a multi-chromatic laser or a multiplicity of laser diodes, each outputting optical signal of a different wavelength.

Gold nanorods have unique size- and shape-dependent optical properties. They have the ability to resonantly absorb and scatter visible and NIR light upon the excitation of their surface plasmon oscillation and usually present intense and narrow absorption/scattering peaks (Jain et al., 2006). Since the Γ(ρ) profile highly depends on the tissue absorption and scattering properties, decorating the tumor with specifically targeted GNR changes the measured Γ(ρ) in the tumor compared with normal tissue. This phenomenon exists as long as the reflected intensity is measured at a wavelength corresponding to the GNR absorption/scattering SPR peak. In the current invention, tissue-like phantoms and mice were irradiated with a 650 nm laser. At this wavelength, certain sizes of GNR can have significant absorption but a negligible scattering coefficient. As a result, the measurements in this work focused on the change in tissue absorption following the GNR injection, rather than on the change in its scattering properties which is mostly measured in NIR molecular spectroscopy and imaging techniques.

According to other embodiments of the invention, the frequency domain photon migration (FDPM) method (Pham et al., 2000; Cerrusi et al., 2011) is used for GNR-based imaging. The FDPM method is a multi-wavelength, high bandwidth (1 GHz) method that has been developed for quantitative and non-invasive measurements of tissue optical and physiological properties (Tromberg et al., 1997). FDPM is used to generate optical absorption and scattering maps at different wavelengths in the NIR region (650-1000 nm), wherein tissue absorption is relatively low and light can penetrate deep volumes of tissue—up to several centimeters (Lin et al., 2011). The detected penetration depth of the photons within the tissue measured by the FDPM method is higher compared to the DR method which measure the reflected intensity only (enabling a detection depth of few millimeters as described in 34 (i)).

The invention will now be illustrated by the following non-limiting Examples.

### Examples

### Materials and Methods

### The Diffusion Reflection Method

The diffusion model (Jacques et al., 2008), as described above, is among the main approaches that best describe the light path in tissues. This approach assumes that light can be treated as a concentration of optical energy that diffuses down a concentration gradient. The loss of energy is caused by the absorbing and scattering components within the tissue (Jacques et al., 2008). The diffusion model can solve several classes of image or property recovery problems. One of the most common among them is the measured Γ(ρ). This Γ(ρ) function, which describes the reflected light intensity (defined as Γ) at the tissue surface in several light source-detector separations “ρ”, presents a strong correlation to the tissue optical properties, such as the absorption coefficient μₐ and the reduced scattering coefficient μₛ′, as discussed above (Schmitt et al., 1990):

Γ(ρ)=[c₁/(ρ)ⁿ]·exp(−μρ).

The Γ(ρ) profile is influenced by the optical properties of the tested tissue, such as its absorption and scattering coefficients (μₐ and μₛ, respectively) and the anisotropy factor “g”. Whereas μₐ is mainly related to tissue's chromophores (Feather et al., 1998), μₛ and g reflect the form and concentration of the scattering components in the irradiated tissue (Hielscher et al., 1997). As the biological tissue is defined as a turbid three-dimensional medium, the scattering property of the tissue is usually defined by the reduced scattering coefficient, μₛ′, calculated by the following equation:

μₛ′=(1−g)μₛ

There are several researches that presented the influence of the tissue's optical parameters on the light path within the tissue. These include the effect of anisotropic optical properties on the photon migration (Dagdug et al., 2003), the time of flight and photon path length for photons in tissues using the radiation transfer equation (Zaccanti et al., 1999) and the penetration depth in irradiated tissue (Bonner et al., 1998).

### Experiments Set I:

### Materials and Methods

### Gold Nanorods (GNR) Fabrication and Targeting

In this experiment, GNR were synthesized using the seed mediated growth method (Nikoobakht et al., 2003). The size, shape, and uniformity of the NGRs were characterized using transmission electron microscopy, and the resultant size was 25 nm×65 nm, with narrow size distribution (10%). A solution of GNR suspended in cetyltrimethylammonium bromide (CTAB) (Sigma-Aldrich, St Louis, Mo.) was centrifuged at 11,000 g for ten minutes, decanted, and resuspended in water to remove excess CTAB. To prevent aggregation, the particles were stabilized in physiological solution, and to improve blood circulation time, a layer of polyethylene glycol (mPEG-SH, molecular weight [MW] 5000 g/mol) (creative PEGWorks, Winston-Salem, N.C.) was adsorbed onto the GNR. This layer also provided the chemical groups that are required for antibody conjugations (SH-PEG-COOH, MW 3400 g/mol). The absorption spectrum of bare GNR, PEGylated and anti-EGFR-coated GNR solutions were measured. Zeta potentials (Maldiney et al., 2011) (ZetaSizer 3000HS, Malvern Instruments, Worcestershire, UK) of the resulting GNR were measured and are presented in the following Table1:

TABLE 1

| Sample | Zeta potential (mV) |
|---|---|
| Bare GNR | +13.1 |
| PEG-coated GNR | +0.87 |
| Anti-EGFR coated GNR | +5 |

The zeta potentials were measured while the GNR were suspended in water with excess cetyltrimethyl ammonium bromide (CTAB).

The zeta potential indicates the stability of colloidal dispersions. With regard to the GNR, the zeta potential refers to the repulsion between adjacent, similarly charged particles. GNR stabilized in CTAB solution showed cationic surfaces (+13.1 mV). This was due to adsorbed CTAB that has a quaternary amine as a hydrophilic head. In contrast, PEG-modified GNR showed a nearly neutral surface (+0.87 mV). To specifically target SCC HNC, the PEGylated GNR were coated with Cetuximab (Erbitux, Merck KGaA, Germany), a monoclonal antibody against EGFRs that is highly sensitive to HNC SCC. The binding of the EGFRs to the GNR was confirmed by zeta potential measurement, resulting in a positive potential (+5 mV, see Table 1). The antibody conjugated GNR were stable for up to 3 months, confirmed by their maintenance of the same plasmon resonance.

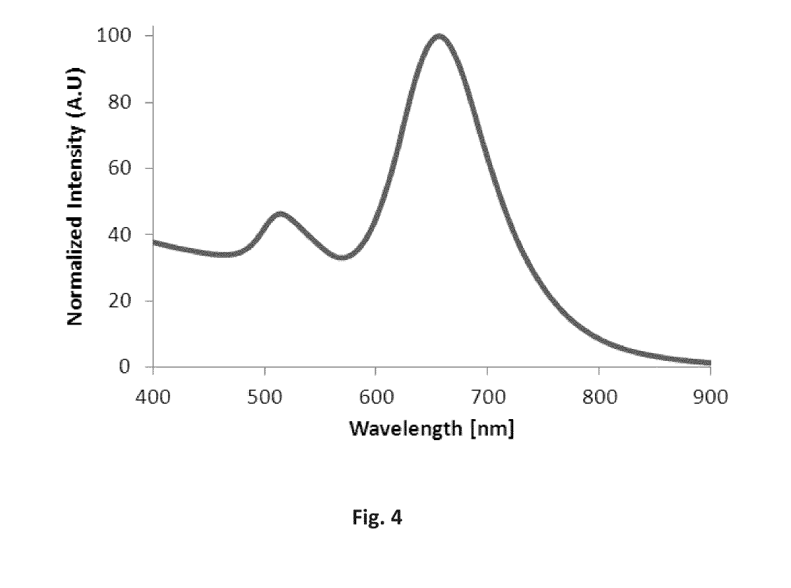

FIG. 4 shows the absorption spectra of GNR for varying wavelengths within the NW range. It is clear form this testing that the best absorption is performed at a wavelength of 650 nm. This preliminary testing was used to determine the optical wavelength to use when irradiating the tissue/phantom in the following experiments.

### System Setup:

For this experiment, the system used 200 was the one described in FIG. 2B. In this system 200 a laser diode based laser device 210 connected to an optical fiber 201 is used to emitting coherent laser beam of 650 nm, which is guided by the optical fiber 201 towards the skin of the patient 10 (in this case a mouse bearing human HNC) for detecting the irradiated light from the cancerous tissue, using a PD 202 detector suitable for detection of light at a wavelength range of the irradiated tissue. The fiber 201 used was 125 μm in diameter and was connected at its output end to a micrometer plate 203 for enabling consecutive reflected light intensity Γ measuring. In this experiment the micrometer plate 203 was moved in twenty incremental steps of 250 μm per step to allow changing the source-detector separation “ρ”, varying between 1-6 mm. The source-detector separation “ρ” is defined, in this experiment, as the distance between the PD 202 location and the light source (i.e. the output of the optical fiber 201 location). The reflected intensity Γ(ρ) (in volts) was collected from the digital scope 220 (Mso/7034a; Agilent Technologies, Santa Clara, Calif.), and the data was processed by using, inter alia, Matlab based analysis algorithms that were developed especially for experiments, systems and methods of the present invention.

### Phantom Preparation:

Solid phantoms with different absorption coefficients were prepared in order to simulate skin tissues with different optical properties (Dam et al., 2001). The phantoms were prepared using India ink 0.1% as an absorbing component, Intralipid® 20% (Lipofundin MCT/LCT 20%, B. Braun Melsungen AG, Melsungen, Germany) as a scattering component (Cubeddu et al., 1997), and 1% agarose powder (SeaKem LE Agarose, Lonza, Norwalk, Conn.) in order to convert the solution into a gel. The solutions were heated and mixed (at a mixing temperature of ˜90° C.) while the agarose powder was slowly added. The absorption spectrum of the India ink was determined using a spectrophotometer, and the absorption coefficient of each phantom was calculated according to the concentration of the ink in each solution. The scattering properties of the phantoms were determined according to the scattering coefficients presented by Cubeddu et al., 1997.

The phantoms were prepared in cell culture plates (90 mm) and were cooled under vacuum conditions (to avoid bubbles). Five phantoms with the same scattering properties and different absorption coefficients were prepared. Each phantom contained 2% of Intralipid and increasing concentrations of India ink: 5.0×10−4, 2.5×10−3, 5.0×10−3, 7.0×10−3, and 1.0×10−2(%). The resulted scattering coefficient was ˜1.6 mm−1, and the resulting absorption coefficients were μₐ=0.0064, 0.0126, 0.0180, 0.0227, and 0.0295 mm−1, respectively. GNR (10 mg/mL) were added into two identical phantom solutions, containing 2×10−3% of ink and 2% of Intralipid (optical properties of μₐ=0.0115 mm−1 and μs′=1.6 mm−1) to achieve final concentrations of 0.03 and 0.008 mg/mL of gold in the phantoms. The solutions were heated and mixed at a temperature of approximately 90° C. while the agarose powder was slowly added. Then, the phantom solutions were poured into a 24-well plate (each well with a 16 mm diameter) and were cooled under vacuum conditions.

### In Vitro Experiment:

A-431 cells (2.5×10⁶) in 5 mL Dulbecco's modified Eagle's medium containing 5% fetal calf serum, 0.5% penicillin, and 0.5% glutamine were divided into two groups for a quantitative cell binding study (each experimental group was run in triplicate). The first group was incubated with 50 μL of anti-EGFR-coated GNR (25 mg/mL) for 30 minutes at 37° C., and the second group (negative control) was incubated under the exact same conditions with anti-rabbit immunoglobulin G (IgG)-coated GNR. After incubation, the medium was washed twice with phosphate buffered saline (PBS) followed by the addition of 1 mL of aqua regia HCl:HNO3 (1:3) (Sigma-Aldrich). After evaporation of the acid, the sediment was dissolved in 5 mL 0.05 M HCl. The gold concentrations of the samples were quantified by atomic absorption spectroscopy (AA 140; Agilent Technologies, Santa Clara, Calif.).

### In Vivo Experiment:

Embodiments of the present invention for tumor detection were evaluated using mice bearing human head and neck cancer (HNC) derived from an A-431 SCC cell line. A-431 cells (2×10⁶) were injected subcutaneously into the back flank area of 10-11-week-old nude mice. These cells express from 2×10⁴ to 2×10⁶ EGFRs per cell (Stanton et al., 1994; Todd et al., 1999). When the tumor reached a size of 7-9 mm in diameter, the mice received 100 μL (25 mg/mL) of immuno-targeted GNR by tail vein injection. Mice tumor and normal tissue (control #1; identical organ on the opposite side, without tumor, after the GNR injection) were scanned immediately after GNR injection and up to ten hours post-injection.

As a control experiment, the same mice were scanned (tumor and normal tissues) before GNR injection (control #2 and #3, respectively). Diffusion reflection measurements were performed on all samples to test the ability to specifically and sensitively detect tumors. All in vivo measurements were performed under appropriate anesthesia: the mice barrier-controlled facility was under the strict care of the veterinarian in charge of the Institutional Animal Care and Use Committee (IACUC).

### Results of Experimental Set I:

### Phantom Results:

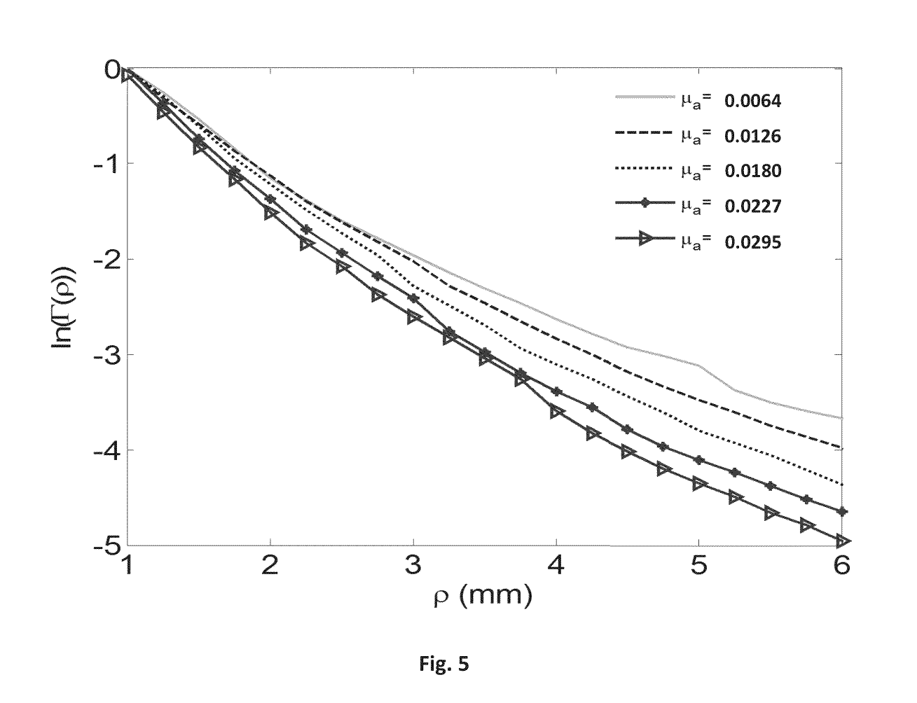

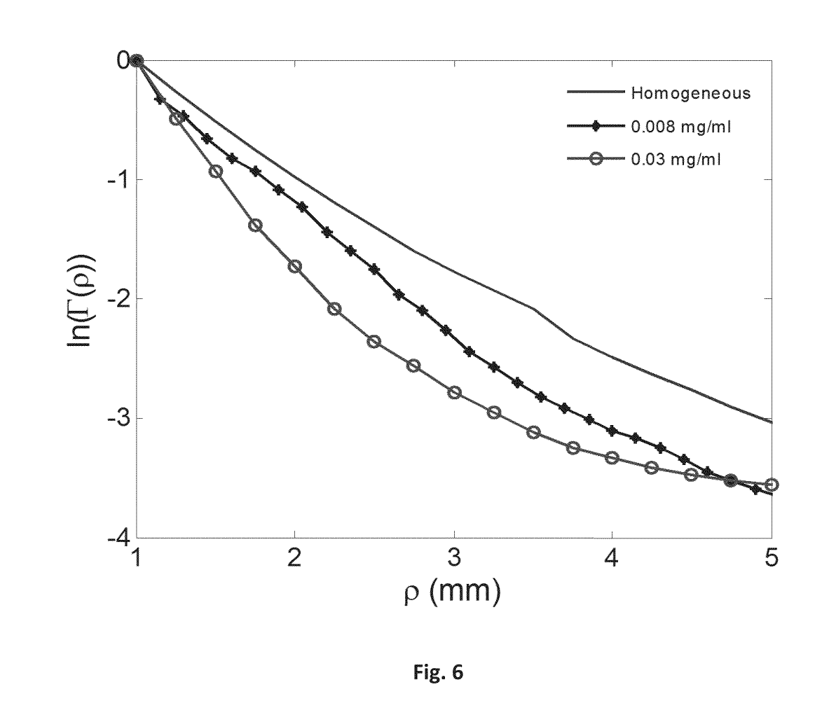

The reflected light intensity from five different phantoms was measured using the experimental setup described above (i.e. in FIG. 2B). Representative results of the reflected light intensity profiles are presented in FIG. 5. The experimental results correlate well with the analytical predictions of the diffusion theory: the larger the absorption coefficient μₐ, the sharper the graph's slope. The phantoms' absorption coefficients were 0.0064, 0.0126, 0.0180, 0.0227, and 0.0295 mm⁻¹, and the slopes represent increasing respective negative values of: 0.57, 0.64, 0.69, 0.74, and 0.81. The increasing negative values of the slopes directly correlate with the increasing concentrations of ink in the phantoms. These results indicate the ability of the system used in the experimental work to clearly distinguish between different absorption coefficients. FIG. 6 presents the reflected light intensity from three solid phantoms as follows: one homogeneous phantom (a solid phantom without GNR, μₐ=0.0115 mm⁻¹ and μₛ′=1.6 mm⁻¹) and two phantoms containing 0.008 and 0.030 mg/mL of GNR. The results clearly indicate that the presence of GNR within the phantom increases the slope of the reflected intensity profile. Moreover, the higher the GNR concentration, the sharper the reflectance graph slope. The phantom with 0.008 mg/mL of GNR represents a negative slope of 1.11, while the phantom containing 0.030 mg/mL of GNR represents a negative slope of 1.39. As mentioned above, GNR have high absorption at 650 nm but negligible scattering properties. Therefore, the observed increase in the graph's slope is due to the increase in the absorption of the irradiated phantom resulting from the presence of the GNR.

### In Vitro Results:

To evaluate the specificity of the interaction between the antibody-coated GNR and the A-431 SCC cancer cells (which highly express the EGFR), two types of GNR were introduced to the cells: the first was specifically coated with anti-EGFR antibody; whilst the second, which was used as a negative control, was coated with a nonspecific antibody (anti-rabbit IgG). Flame atomic absorption spectroscopy measurements quantitatively demonstrated that the active tumor targeting (anti-EGFR-coated GNR) was significantly more specific than the control experiment (anti-rabbit IgG coated GNR). The A-431 cells took up 21.8±2.3 μg of targeted GNR, whilst parallel cells in the negative control experiment absorbed only 0.20±0.01 μg of GNR (Reuveni et al. 2011). These results correlate well with previously published studies, which report that head and neck SCC express from 2×10⁴ to 2×10⁶ EGFRs per cell (Stanton et al 1994).

### In Vivo Results:

The tumor-bearing mice were irradiated, under appropriate anesthesia, and the reflected light intensity was measured using the optical setup described in FIG. 2B.

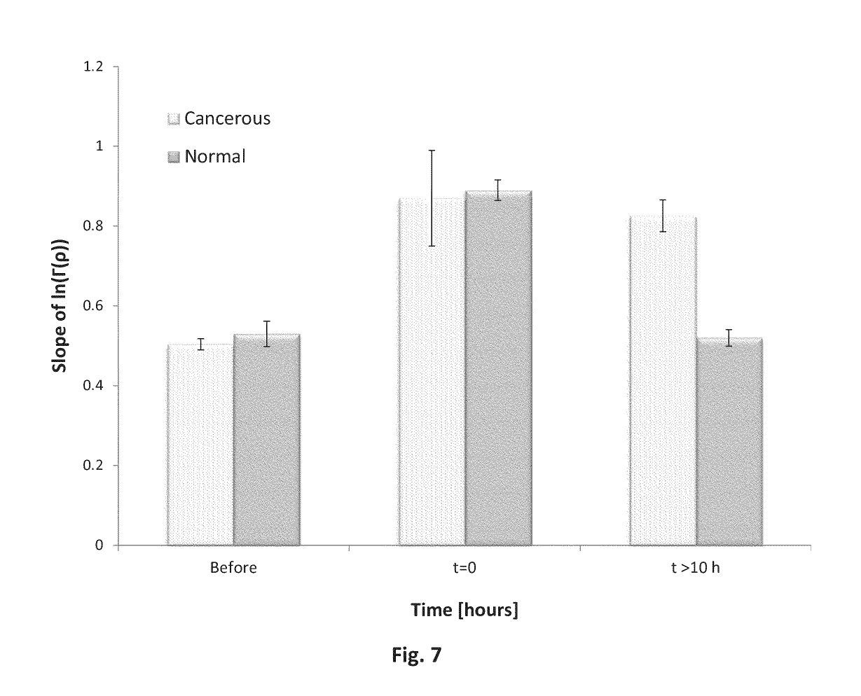

The reflectance measurements were performed before the GNR injection and for several delay times (15 minutes, 3, 5, and over 10 hours) post-injection. The slopes of the reflected light intensity profiles were calculated, and average results are shown in FIG. 7.

FIG. 7 compares the reflected light intensity slopes (absolute values) of the cancerous and the normal tissues, for three representative times: (1) before GNR injection (control #2 and #3), (2) immediately (˜15 minutes) after intravenous injection, and (3) more than ten hours post-injection. It is clearly demonstrated that ten hours post GNR injection there is a significant change (of more than 60%) between the reflectance profiles of the cancerous and the normal tissue (control #1).

This change results from specific accumulation of GNR in the tumor. It is also demonstrated that immediately after GNR injection, as well as for the delay times of three and five hours post-injection (results not shown), the reflectance profiles of both the cancerous and the normal tissues present an increase in their slopes, which indicates the GNR's long circulating time in the blood. After that time, the GNR were gradually cleared from the blood until their complete clearance from the normal tissue, resulting in a decrease of its reflectance slope compared with the cancerous tissue, which kept a stable value of 0.8. Regarding control #3 (normal tissue before the GNR injection), the mice's normal tissues were irradiated in different areas in the mice's skin tissue, and the reflectance slopes of the different areas were almost identical, resulting in a small standard deviation (small error bar in the left column in FIG. 7). This high similarity of these slopes indicates that any non-cancerous area of the skin can be irradiated, and the resulted reflection slope will always be lower than the tumor reflection slope ten hours or more post the GNR injection, enabling consistent tumor detection.

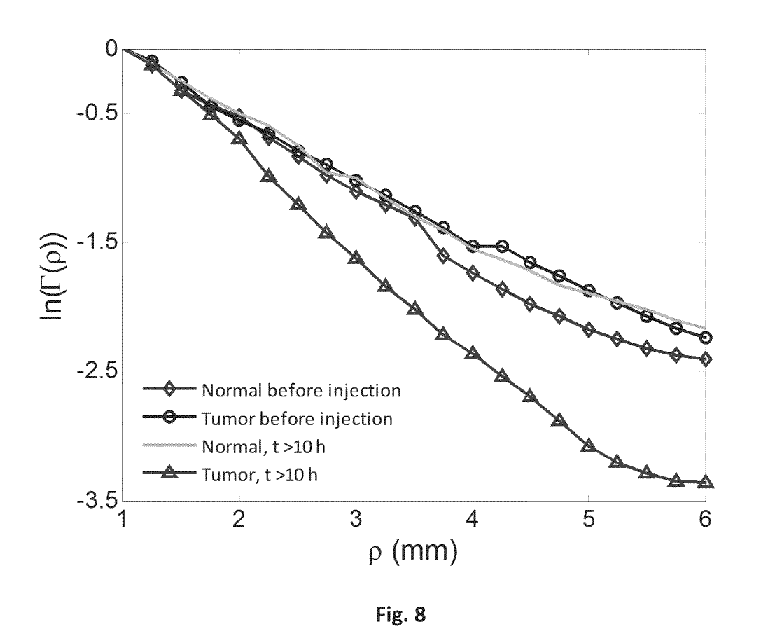

FIG. 8 emphasizes the difference between the slopes before GNR injection and more than ten hours post-injection for the cancerous and normal tissues, as directly obtained from the reflected light intensity measurements. While the reflectance slope, which directly indicates the absorption coefficient of the normal tissue, is the same before GNR insertion and more than ten hours post-injection, the tumor clearly represents a sharper slope. This clear discrimination between cancerous and normal tissue enables sensitive and specific cancer detection based on diffusion reflection measurements.

### Experiments Set II:

### Materials and Methods

### The Optical Setup:

In this experiment double-wavelength measurements were performed, where the first wavelength correlates with the absorption peak of the suspended GNR and the second wavelength correlates with the expected extension and red-shift (Δλ) of the GNR's absorption spectrum. For this purpose, a noninvasive optical technique was designed and built for reflected light intensity measurements. The system setup is similar to that described in FIG. 2B.

The set-up included two laser diodes, with wavelengths of 650 nm and 780 nm, which were optically bundled to a split fiber (125 mm in diameter) for irradiation. A portable photodiode, deposited at different distances ρ on the samples' surface, served as a detector, enabling DR intensity (Γ) measurements in several light-source detector separations (Γ(q)). The photodiode's cross-section diameter was 1 mm² The initial distance ρ between the light source and the first location of the photodiode was approximately 1 mm. A micrometer plate, to which the optic fiber was attached, enabled a consecutive reflected light intensity measurement. The micrometer plate was moved in 21 incremental steps of 250 μm each. The reflected light intensity was collected from 1 mm (the initial distance between the light source and the photodiode) to 6.25 mm. The reflected intensity Γ(ρ) (in Voltage) was collected using the digital scope (Agilent Technologies, Mso7034a, Santa Clara, Calif.) as well as a DAQ (USB-6008, National Instruments, Israel). The data was processed using the MATLAB (the Mathworks Inc., 2010) and LabView (National Instruments, 2009) softwares.

### Gold Nanorods Fabrication:

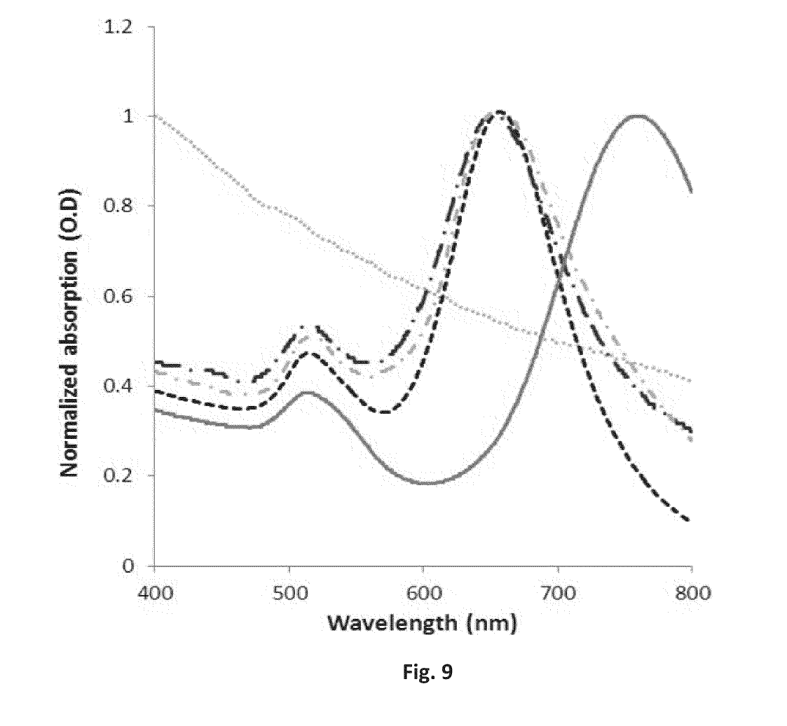

Two sizes of GNR types, presenting absorption spectra in 650 and 780 nm, were prepared and the DR method ability to distinguish between their different SPR values was proved. The GNR were synthesized using the seed mediated growth method (Nikoobakht et al., 2003). Their size, shape and uniformity were characterized using transmission electron microscopy (TEM) (FIG. 9) and presented a narrow size distribution (10%). The absorption spectra of the GNR solutions were measured and are presented in FIG. 9. Two kinds of GNR were synthesized: the first, named as GNR₆₅₀, have average dimensions of 65×25 nm, resulting in an aspect ratio “R” of 2.6 and an average effective radius “r_{eff}” of 19 nm (Jain et al., 2006). These GNR presented an absorption peak at 650 nm. The second kind of GNR, named as GNR₇₈₀, presented average dimensions of 52×13 nm, resulting in R=4; r_{eff}=12.5 nm and an absorption peak at 780 nm According to Jain et al., 2006, these GNR have high absorption properties at 650 nm and 780 nm but much less dominant scattering properties: while GNR₆₅₀ are expected to have an absorption coefficient three times higher than their scattering coefficient, GNR₇₈₀ are expected to present an absorption coefficient that is about 14 times higher than their scattering coefficient.

Only GNR₆₅₀ were used for the in-vivo measurements in order to illuminate with 780 nm laser, according to the expected spectral red-shift.

### Solid Phantoms Preparation:

Solid phantoms were prepared and simulated the skin tissue optical properties (Dam et al., 2001). The phantoms were prepared using 3×10⁻³% of India Ink, as an absorbing component, 2% of Intralipid (Lipofundin MCT/LCT 20%, B. Braun Melsungen AG, Germany) as a scattering component (Cubeddu et al., 1997) and 1% Agarose powder (SeaKem LE Agarose, Lonza, USA), in order to convert solution into a gel. We determined the absorption spectrum of the India ink (see FIG. 9, dotted line) using a spectrophotometer and calculated the absorption coefficient μₐ of each phantom according to the concentration of the ink in each solution. The scattering properties of the phantoms were experimentally determined in our previous work. The resulted μₐ of the phantoms was 0.0137 mm⁻¹ and the reduced scattering coefficient μ′ₛ was ˜1.45 mm⁻¹.

Into six identical phantom solutions, GNR₆₅₀ (4 mg/mL) were added to achieve final concentrations of 0.01, 0.02, 0.05, 0.1, 0.15 and 0.2 mg/ml of gold. In addition, GNR₇₈₀ (4 mg/mL) were added to another phantom and presented a final concentration of 0.02 mg/ml.

All phantom solutions were heated and mixed at a temperature of approximately 90° C. while the Agarose powder was slowly added. All phantom solutions were poured into a 24 wells plate (each well of a 16 mm diameter) and were cooled under vacuum conditions (to avoid bubbles).

### Dark Field Reflectance Imaging:

Dark field reflectance images of GNR650 were captured using the hyper spectral imaging system (Nuance, CRi, MA, USA). A Xenon illumination, along with a 40× dark field objective (0.75 NA) and 32-bit ultrasensitive CCD camera detector (N-MSIEX, CRi, MA, USA) were used for imaging in RGB (red green blue) mode. Microscopy then was performed on a Nikon 80i Microscope (Nikon instruments, Inc). Images were acquired using the Nuance software version 2.1. In dark field microscopy, a very narrow beam of white light is delivered on top of the sample. The large scattering angle allows detection of highly scattering objects (such as GNR, due to their enhanced SPR) with a very little background signal. We prepared three different concentrations of GNR₆₅₀ solutions as three volumes of 5, 12 and 20 μl were taken from a solution presenting 3.1 mg/ml of GNR₆₅₀. The resultant densities of the GNR₆₅₀, on slides with dimensions of 1 cm², were 0.0155, 0.0372 and 0.062 mg/cm².

### In Vivo Experiment:

In-vivo DR measurements were evaluated using mice bearing human HNC derived from an A-431 SCC cell line. A-431 cells (2×106) were injected subcutaneously into the back flank area of 10-11 week-old nude mice. Two concentrations of GNR₆₅₀ were injected into two groups of mice: group 1 received 200 ml of ˜10 mg/ml while group 2 received 200 ρ 1 of ˜30 mg/ml. When the tumor reached a size of five to seven millimeters in diameter, the mice received the GNR₆₅₀ by tail vein injection. Mice tumor and normal tissue were scanned before GNR₆₅₀ injection and ˜16 hours post injection. Diffusion reflection measurements were performed on three to five different sites on the mice's skin.

All in-vivo measurements were performed under appropriate anesthesia: the mice barrier-controlled facility was under the strict care of the veterinarian in charge of the Institutional Animal Care and Use Committee (IACUC). The mice were inspected daily by the veterinarian, who handles the appropriate tests and treatment protocols, as required. All research protocols were followed closely by the veterinarian. All major procedures were performed in the surgical facilities using general anesthesia and standard, aseptic surgical techniques.

In this line of experiments it was shown that the key advantage of the DR imaging based detection is that it correlates the absorber's molecules concentration and the irradiated light intensity Γ(ρ). This correlation can be used for identifying the tumor's size.

### Results of Experiments Set II:

### DR Measurements of Solid Phantoms Containing GNR:

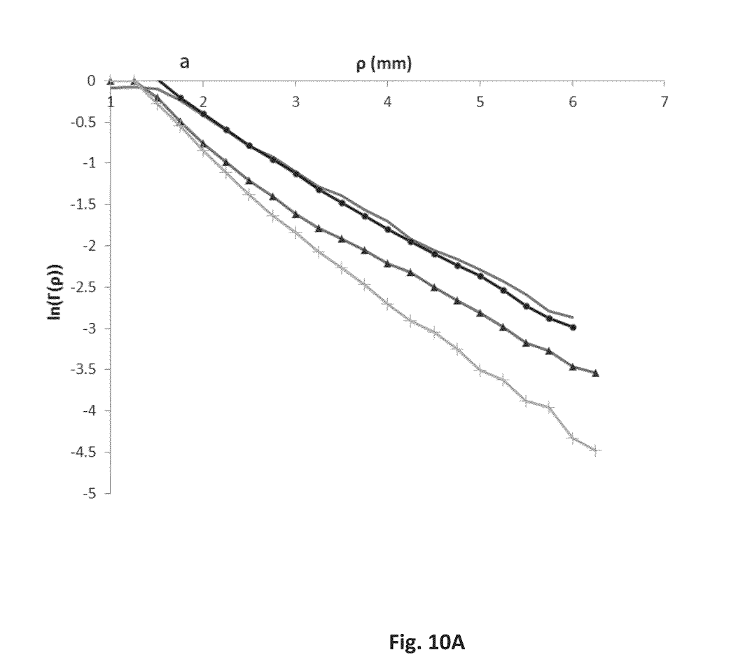

DR measurements of solid phantoms containing both, GNR₆₅₀ and GNR₇₈₀, were performed using the experimental set-up described above. Representative results of the reflected light intensity profiles of a phantom with 0.01 mg/ml of GNR₆₅₀ are presented in FIG. 10A. The experimental results correlate well with the predicted behavior: first, the solid phantom without GNR (named as a homogeneous phantom) presents a DR profile with a more negative slope following 650 nm illumination compared to 780 nm illumination (the slopes were 0.69 and 0.60 for the triangle marked line and solid line, respectively). This is in a good correlation with the ink absorption spectrum presented in FIG. 9, which shows a higher absorption in the 650 nm. While the 650 nm illumination results in a more negative slope (of 0.87, the cross marked line in FIG. 10A) than the homogeneous phantom, the phantom that was illuminated with 780 nm kept a constant slope before and after the 650 nm illumination.

FIG. 10B presents similar results for 780 nm illumination of a phantom containing 0.02 mg/ml of GNR₇₈₀. While the DR curve following 780 nm illumination presented an increase in its DR slope compared to the phantom without GNR (the slopes values of the circle marked and solid lines were 0.79 and 0.60, respectively). The DR curve following 650 nm illumination remained the same (a slope of 0.63, the cross marked line in FIG. 10B). As mentioned above, these GNR present high absorption properties at 650 and 780 nm but much less dominant scattering properties. Therefore, the observed increase in the graphs' slopes is due to the increase in the absorption of the irradiated phantom, resulting from the presence of the GNR. These results suggest that our detection method can observe different sizes of GNR (based on their different SPRs).

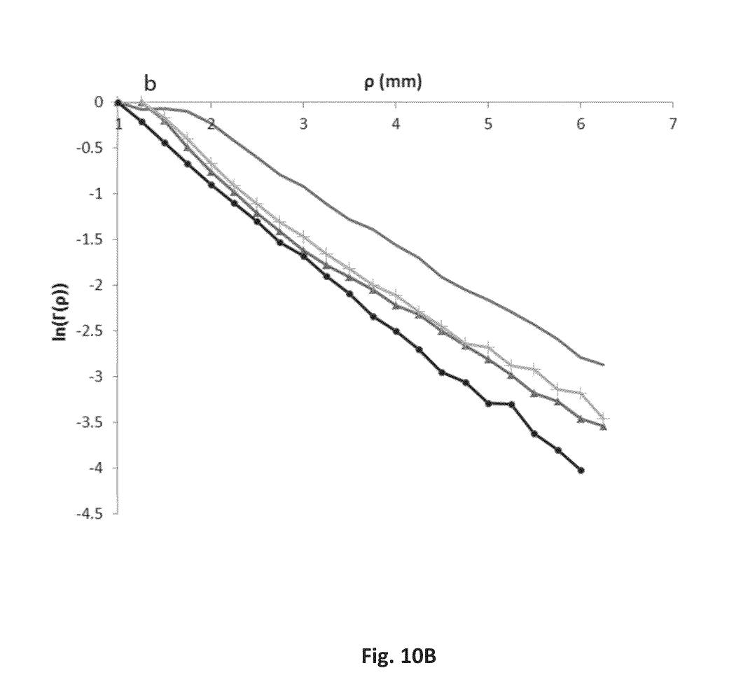

FIG. 11 presents the Δ slopes of all irradiated phantoms, calculated from their DR profiles. The Δ slope is defined as the difference between the DR slopes of a phantom with GNR₆₅₀ and a homogeneous phantom. It is well seen that for low GNR concentrations (0.01 and 0.02 mg/ml in FIG. 11) the DR profiles present the predicted behavior, as the Δ slopes present significant values following 650 nm illumination only (0.19±0.02 and 0.34±0.05 for 0.01 and 0.02 mg/ml, respectively). Starting from 0.05 mg/ml, the Δ slopes following 780 nm illumination became significant, resulting in 0.45±0.04, 0.88±0.21, 1.3±0.18 and 1.31±0.27 for 0.05, 0.1, 0.15 and 0.2 mg/ml of GNR₆₅₀, respectively. The resulted Δ slopes following 650 nm illumination present similar values of 0.58±0.2, 0.75±0.21, 1.29±0.18 and 1.19±0.27 for the same concentrations, respectively. This similarity in the Δ slopes following both wavelengths irradiation, despite the fact that the phantoms contained GNR₆₅₀ only, indicates that a red-shift and peak expansion occurred in the GNR absorption spectrum. In order to identify this spectral red-shift, dark-field microscopy was used.

### In Vitro Dark Field Reflectance Imaging of Different GNR Concentrations:

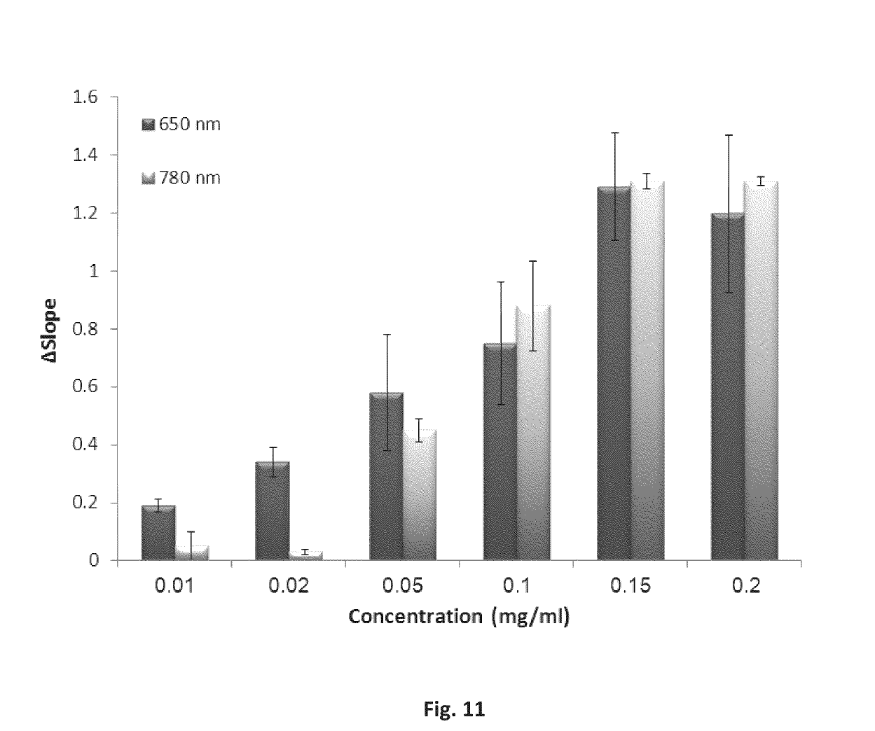

Different concentrations of GNR₆₅₀ were measured using the dark-field microscopy and their total absorption spectra were collected, according to the description in the Materials and Method section of Experiment 2, discussed above. The resulted absorption spectra of two different densities, 0.0155 and 0.0372 mg/cm², are presented in FIG. 12. The dashed curve shows the absorption spectrum of GNR₆₅₀ in a relatively low density on the slide, of 0.0155 mg/cm². This spectrum well correlates the absorption properties of the GNR₆₅₀ suspended in water (the dashed line in FIG. 9), presenting a colloidal suspension with high spacing between particles, therefore no red-shift is observed (Δλ=0). The dashed-dotted curve in FIG. 12 is the resulted absorption spectrum of GNR₆₅₀ with a higher density of 0.037 mg/cm². The absorption spectrum still presents the GNR “fingerprint” peak in 530 nm, yet the SPR coupling of the GNR₆₅₀ is well observed as the intense absorption peak shifted to the red region, resulting in an absorption peak of 733 nm (Δλ=83 nm). A larger red shift (to approximately 750 nm, Δλ=100 nm) was observed for GNR₆₅₀ density of 0.062 mg/cm² (data not shown). These results indicate that in high densities of GNR₆₅₀, SPR coupling occurs. It explains the increase in the DR slopes of phantoms with high GNR₆₅₀ concentrations following 780 nm illumination.

Still, since the GNR are not homogeneously dispersed on the slide, the total spectrum should include different SPR peaks, of 650 nm and of several red shifted peaks toward the 750 nm Indeed, the dotted line in FIG. 12 shows a broaden graph which was also observed in high densities of GNR₆₅₀ (0.037 and 0.062 mg/cm2). The broadening indicates an inhomogeneous dispersion of the nanoparticles, resulted in an ensemble of red shifts (as was also previously presented by Mallidi et al., 2009). Since the DR slopes of phantoms containing highly concentrated GNR₆₅₀ increased following both, 650 nm and 780 nm illuminations, the possibility that this broadening in the absorption spectrum result from a change in the refractive index of the GNR within the Intralipid surrounding or the tissue was also tested. Simulations and other research were done and suggested that the refractive index of the phantom or tumorous tissue surrounding the GNR do not influence the GNR₆₅₀ SPR. Thus it can be deduced that Δ slopes presented in FIG. 11, result from the SPR coupling that occurred in the phantoms that contained high concentrations of GNR₆₅₀.

### In Vivo DR Measurements of Tumor-Bearing Mice:

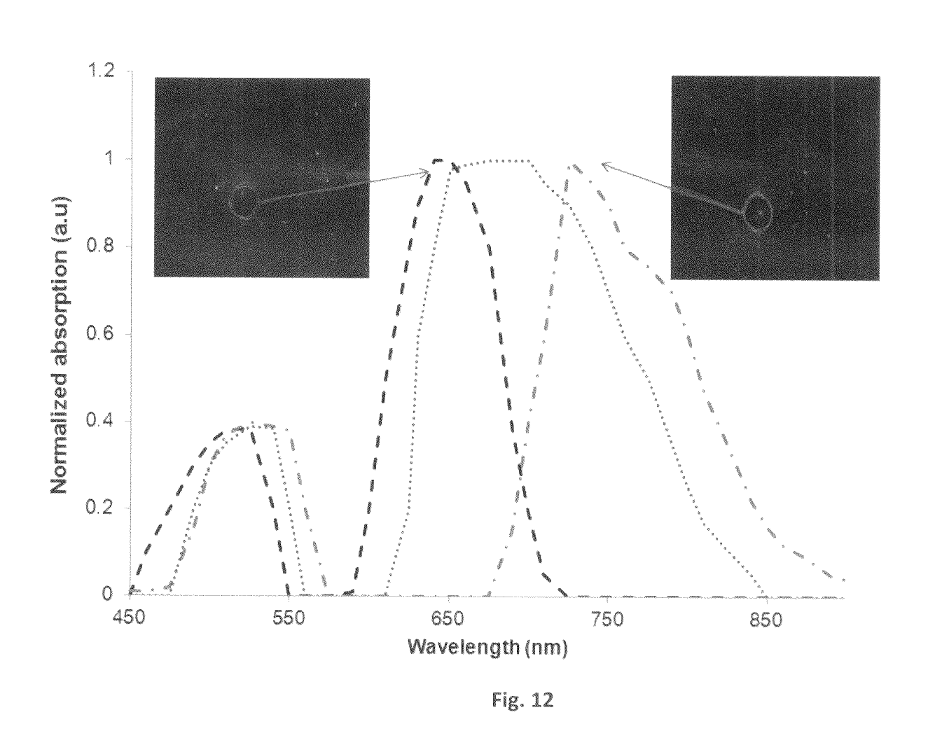

The tumor-bearing mice were irradiated, under appropriate anesthesia, and the reflected light intensity was measured using the optical set-up described above. The reflectance measurements were performed before the GNR₆₅₀ injection and approximately sixteen hours post-injection. The slopes of the reflected light intensity profiles were calculated and representative results are shown in FIGS. 13A and 13B. FIG. 13A presents the DR profiles of the tumor bearing mice, group 1. As was mentioned in the Materials and methods section relating to experimental work of experiment 2, this group received a relatively low concentration of GNR₆₅₀. Indeed, the DR spectra presents the same behavior observed for phantoms containing low concentrations of GNR₆₅₀ as the slope of the curve increased following 650 nm illumination only (0.50±0.014 and 0.67±0.02 before and 16 hours post injection, solid and triangle marked lines, respectively), while illumination with 780 nm did not affect the DR slope (an average slope of 0.46±0.042, circle and asterisk marked lines, before and 16 hours post injection, respectively).

In contrast, the DR spectra of group 2, shown in FIG. 13B, present the behavior observed for highly concentrated GNR₆₅₀ in phantoms, as the DR curves show an increase in their slope following both 650 nm (triangle marked line) and 780 nm (asterisk marked line) illuminations compared to their slopes before illumination (solid and circle marked lines, respectively). The average slopes increased from 0.55±0.032 to 0.8±0.009 before and 16 hours post illumination, respectively. These results well indicate that DR measurements can identify a red-shift in tumors in vivo in real time.

Spatial diffusion reflection measurements with GNR as contrast agents is based on the change in the absorption properties of the tumor site following intravenous injection of EGFR targeted GNR. In this above-described experiment 2, the spectral red-shift occurs in high concentrations of GNR was suggested as an additional parameter for DR-based tumor detection measurements.

In the above study, it is well observed that the higher the GNR₆₅₀ concentration, the more intense is the Δ slope following 780ₙₘ illumination. In order to verify whether the observed red-shift of the GNR₆₅₀ in phantoms resulted from SPR coupling only, and not from the difference between the refractive indexes of the water and the phantom, the discrete dipole approximation (DDA) method (Draine et al., 2004) was used.

The results suggested that a very small red-shift of Δλ, of approximately 10 nm, is expected for the GNR₆₅₀ in phantoms or tissues compared to GNR₆₅₀ suspended in water. The expected spectral red-shift of different concentrated GNR₆₅₀ was calculated from in vitro measurements, using the dark-field microscopy. The results, shown in FIG. 12, present a spectral red-shift of Δλ=83 nm in high densities of GNR₆₅₀. This shift was observed while a single GNR (each surrounded by other GNR) was detected. As for a group of GNR, an expansion of the absorption peak was observed, indicating an inhomogeneous dispersion of the GNR.

It was also exhibited (e.g. in FIG. 11) that a broadening in the absorption spectra, as in high concentrations of GNR₆₅₀, the DR slope increased almost identically following both, 650 nm and 780 nm illuminations. If only a red-shift occurred, the DR slopes should introduce an increase in the DR slopes following 780 nm illumination only.

FIGS. 13A and 13B present real-time DR measurements of two groups of tumor bearing mice. The two groups presented different GNR₆₅₀ concentrations in tumor 16 hours post GNR₆₅₀ injection. The results indicate a behavior similar to the observed behavior in FIG. 11: the DR measurements of group 1, which received a low concentration of GNR₆₅₀, showed an increase in their slopes following 650 nm illumination only, indicating that no red-shift occurred in the accumulated GNR in tumor. In contrast, DR measurements of group 2 presented an increase in the DR slope following both, 650 and 780 nm illuminations, suggesting a broadening of the SPR toward the red wavelengths range.

Intravenous administration of targeted GNR results either in a specific binding between the EGFR-targeted GNR and the cancer cells (like SCC), or the inevitable non-specific distribution in the blood and other organs. Further investigation is required in order to demonstrate our ability to distinguish between non-specific and specific (targeted) binding of the functionalized nanoparticles based on their spectral shift. Targeted GNR will present a Δλ in the tumor site due to their specific attachment to the HNC cells resulting in an inter-particle coupling effect. If there are no cells of interest in the sample, the measured resonant wavelength peak will be the same as of a single GNR. Thus, by screening the skin tissue with a double-wavelength DR set-up, the tumor can be detected.

In conclusion, this section demonstrated in tissue-like phantoms and in vivo mice model that DR imaging can detect the spectral red-shift and the peak broadening that occur in highly concentrated GNR.

### Experiments Set III:

### Materials and Methods

Monte Carlo Simulation of Reflected Light Intensity from Irradiated Tissues:

In order to substantiate and extend the experimental results, a MC simulation of photon migration within irradiated tissues was built. The simulated tissues presented optical properties that were chosen according to skin optical properties (Dam et al., 2001; Cubeddu et al., 1997). A constant reduced scattering coefficient μ′ₛ=1:6 mm⁻¹ and varying absorption coefficients, μₐ=0:0115, 0.0126, 0.0182 and 0.0227 mm⁻¹, were used. The absorption coefficients just slightly differed from each other, in order to test the sensitivity of the reflected light profiles to different absorption properties of the tissue. The main assumptions of the simulations were as follows:

(i) A turbid three dimensional medium was defined according to the scattering coefficient μₛ, anisotropy factor g, layer width L and a changing absorption coefficients ρₐ. L was set to be 1 meter, much larger than a photon step “dr”. As will be described hereinafter, L>>dr), thus the tissue can be considered infinite. The reduced scattering coefficient μ′ₛ was calculated by the following equation:

μ′=(1−g)μₛ.

(ii) Photons were launched, without reflection, perpendicular to the surface, into a single point on the lattice plane x=y=z=0.

(iii) For each photon, with a given location (x_{old}, y_{old}), z_{old}) and a propagation direction (θ_{old}, φ_{old}), the direction (θ_{new}, φ_{new}) after a step of dr=250 μm was calculated according to the scattering and absorption properties, as follows: (3.1) The probability of a photon to survive was determined by exp(−μₐ·dr). (3.2) The probability of a photon to scatter was [1−exp(−μₐ·dr)]. If the photon scattered, its new direction was calculated: θ_{new}=θ_{old} s₁·s₂·cos(g) and φ_{new}=φ_{old}+(1−s₁)·s₂·cos(g). While s₁ is a random number from the group {0, 1} and s₂ is a random number from the group {−1, 1}.

(iv) If the photon survived, the new location (x_{new}, y_{new}, z_{new}) was calculated using:

x_{new}=x_{old}+dr·sin θ_{new}·cos φ_{new}; y_{new}=y_{old}+dr·sin θ_{new}·sin φ_{new}; z_{new}=z_{old}+dr·cos θ_{new}.

(v) When photons returned to the surface z=0 they were emitted from the system. The locations in which the photons reached the lattice surface (x, y, 0) were saved. The simulation displayed the radial distribution of reflected photons around the injection point to perform simulated ln(ρ²Γ(ρ)) graphs for the different absorption coefficients.

### The Experimental Set-Up:

A noninvasive optical technique was designed and built similar to that described above, in respect to FIG. 2B. The setup included a laser diode with a wavelength of 650 nm as an excitation source. The choice of this wavelength is due to its safety and proximity to the NIR region, in which light can more easily penetrate the tissue. The irradiation was carried out using an optic fiber with a diameter of 125 μm to achieve a pencil beam illumination. We used a portable photodiode as a photo detector. The photodiode was deposited in different distances (source-detector separations) ρ on the sample surface in order to enable Γ(ρ) measurements. The photodiode had a cross-section diameter of 1 mm² and was kept in close contact with the tissue surface to avoid ambient light from entering the detection system and to avoid potential light loss through the specimen edges. The initial source-detector separation ρ between the light source and the first photodiode was approximately 1 mm.

A consecutive reflected light intensity measurement was enabled using a micrometer plate which was attached to the optical fiber. The micrometer plate was moved by incremental steps of 250 μm each. As a result, the reflected light intensity was collected from 21 source-detector distances with ρ varying between 1 mm (the distance between the light source fiber output and the first photodiode) and 6.25 mm. The reflected intensity Γ(ρ), presenting units of Volt per mm, was collected using a digital scope (Agilent Technologies, Mso7034a, Santa Clara, Calif.) and the data was processed using MATLAB designated software.

### Solid Phantoms:

Solid phantoms with different absorption coefficients were prepared in order to simulate skin tissues with different optical properties (Dam et al., 2001). The phantoms were prepared using varying concentrations of India ink 0.1%, as an absorbing component and a constant concentration of Intralipid (IL) 20% (Lipofundin MCT/LCT 20%, B. Braun Melsungen AG, Germany), as a scattering component (Cubeddu et al., 1994). Agarose powder 1% (SeaKem LE Agarose, Lonza, USA) was added in order to convert solution into gel. The absorption spectrum of the India ink was determined using a spectrophotometer and the absorption coefficient of each phantom was calculated according to the concentration of the ink in each solution. All phantoms presented the same scattering properties using 2% of IL (this concentration refers to the solid fraction in the examined solution). The phantoms were prepared in cell culture plates (90 mm) and were cooled in vacuum conditions (to avoid bubbles). The phantoms' solutions were stirred continuously (except for the period in which they were solidified in vacuum) in order to obtain high uniformity.

The optical properties of the irradiated solid phantoms are presented in the following Table 2. Eight different phantoms were prepared. The ink and IL concentrations, as well as the resultant absorption properties of the phantoms, are presented in Table 2. The concentration of IL refers to the fraction of solids in the solution while the concentration of ink pertains to the fraction of the original product.

TABLE 2

| | Ink | Intralipid | Absorption |
|---|---|---|---|
| | concentration | concentration | coefficient μₐ |
| | [%] | [%] | [mm⁻¹] |
| 1. | 1 × 10⁻³ | 2 | 0.0092 |
| 2. | 1.5 × 10⁻³ | 2 | 0.0104 |
| 3. | 2 × 10⁻³ | 2 | 0.0115 |
| 4. | 2.5 × 10⁻³ | 2 | 0.0126 |
| 5. | 3 × 10⁻³ | 2 | 0.0137 |
| 6. | 4 × 10⁻³ | 2 | 0.016 |
| 7. | 5 × 10⁻³ | 2 | 0.0182 |
| 8. | 7 × 10⁻³ | 2 | 0.0227 |

In addition, GNR (3.1 mg/mL) were added into six phantom solutions, containing different ink concentrations but a constant concentration of IL, as shown in Table 3 below, which shows GNR, Ink and IL concentrations in the phantoms used. Small quantities of GNR were added to the Ink and IL phantoms solutions in order to test the DR method sensitivity to small GNR concentrations.

TABLE 3

| GNR | Ink | Intralipid |
|---|---|---|
| Concentration | concentration | concentration |
| [mg/ml] | [%] | [%] |
| 0.002 | 1.5 × 10⁻³ | 2 |
| 0.003 | 1.5 × 10⁻³ | 2 |
| 0.006 | 1.5 × 10⁻³ | 2 |
| 0.008 | 3 × 10⁻³ | 2 |
| 0.02 | 2 × 10⁻³ | 2 |
| 0.088 | 1.5 × 10⁻³ | 2 |

The solutions were heated and mixed at a temperature of approximately 90° C. while the agarose powder was slowly added. The phantom solutions were then poured into cell culture plates (90 mm) and cooled under vacuum conditions.

### GNR Fabrication and Targeting:

GNR were synthesized using the seed mediated growth method (Nikoobakht et al., 2003). Their size, shape and uniformity were characterized using transmission electron microscopy (TEM) and the resultant shape was 25 nm×65 nm, with narrow size distribution (10%). In order to prevent aggregation, to stabilize the particles in physiological solutions and to improve blood circulation time, a layer of polyethylene glycol (mPEG-SH, MW 5,000 gr/mol) (creative PEGWorks, Winston Salem, USA) was adsorbed onto the GNR. This layer also provided the chemical groups that are required for antibody conjugations (SH-PEG-COOH, MW 3,400 gr/mol). A solution of GNR suspended in cetyltrimethylammonium bromide (CTAB) (Sigma Aldrich, USA) was centrifuged at 11,000 g for 10 minutes, decanted and resuspended in water to remove excess CTAB. 200 μl of mPEG-SH (5 mM) (85%) and SH-PEG-COOH (1 mM) (15%) were added to 1 ml of GNR solution. The mixture was stirred for 24 hours at room temperature and was dialyzed for three days. The absorption spectrum of PEGylated GNR solution presented a strong peak in 650 nm. The cell targeting was performed using the heterofunctional PEG that was covalently conjugated to the anti-EGFR monoclonal antibody Cetuximab (Erbitux, Merck KGaA, Germany), known to specifically target SCC HNC tumors and to be non-toxic in therapeutic concentrations in humans (Baselga, 2001). The antibody conjugated GNR were stable for up to three months, confirmed by their maintenance of the same plasmon resonance. Also, we found that the PEGylated GNR were highly stable. Zeta-potentials (Maldiney et al., 2011) (ZetaSizer 3000HS, Malvern Instruments, UK) of the bare GNR showed cationic surfaces (+40 mV), while the PEGylated GNR showed a nearly neutral surface (−0.5 mV).

### In Vivo Experiment:

The method of the invention for tumor detection was evaluated using mice bearing human HNC derived from an A-431 SCC cell line. A-431 cells (2×10⁶) were injected subcutaneously into the back flank area of 10-11 week-old nude mice. When the tumor size reached a size of approximately 9 mm in diameter, the mice received 100 μL, (25 mg/mL) of immuno-targeted GNR via tail vein injection. The mice tumor was scanned immediately after GNR injection and up to ten hours post-injection.

All in vivo measurements were performed under appropriate anesthesia: the mice barrier controlled facility was under the strict care of the veterinarian in charge of the Institutional Animal Care and Use Comitee (IACUC). The mice were inspected daily by the veterinarian, who handles the appropriate tests and treatment protocols, as required. All research protocols were followed closely by the veterinarian. All major procedures were performed in the surgical facilities using general anesthesia and standard, aseptic surgical techniques.

### Flame Atomic Absorption Spectroscopy:

The Flame Atomic Absorption (FAA) (AA 140; Agilent Technologies, Santa Clara, Calif.) spectroscopy was used for the evaluation of the GNP concentration in tumor. The tumor was dissolved in Aqua Regia HCl:HNO3 (1:3) (SigmaAldrich, USA) and the resulting solution was warmed to a temperature of 70° C. until the total evaporation of the Aqua Regia. The suspension was then dissolved in 10 ml HCl 0.05M. The HCl acid was filtered using a 0.45 μm pore size syringe filter (Miller-GC, Millipore Irland LTD, IRL) to remove tissue residues. The filtered HCl solution absorbance was determined using the FAA spectroscopy. The sample was introduced into the flame by conventional aspiration.

### Results of Experiments Set III:

### Simulation Results:

Simulations of the reflected light intensity from tissues with different optical properties were performed according to the description in the Materials and Methods section above. Photons penetrated and advanced randomly in the tissue. Several absorption coefficients were considered and the resultant logarithmic graphs of the reflected light intensity are shown in FIG. 2(a).

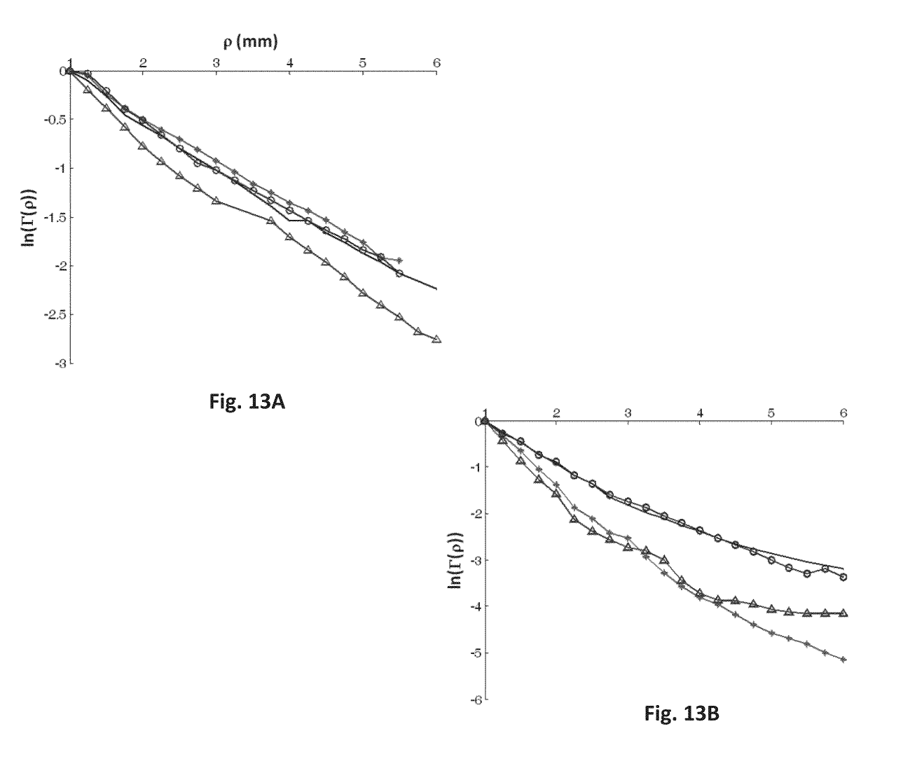

The simulation results in FIG. 14 present the predicted dependence of the reflected light intensity profile on the lattice absorption coefficient: the higher the absorption coefficient, the sharper the decay of the reflected light intensity profile. This is in agreement with the Γ(ρ) equation: Γ(ρ)[c1/ρⁿ]exp(−μρ), in which the absorption coefficient is presented in the exponential decay term. Furthermore, despite the small differences between the absorption coefficients the slopes still differ from each other, resulting in: 0.03, 0.033, 0.054 and 0.084 for the absorption coefficients μₐ of: 0.0115 mm⁻¹, 0.0126 mm⁻¹, 0.018 mm⁻¹ and 0.0227 mm⁻¹, respectively.

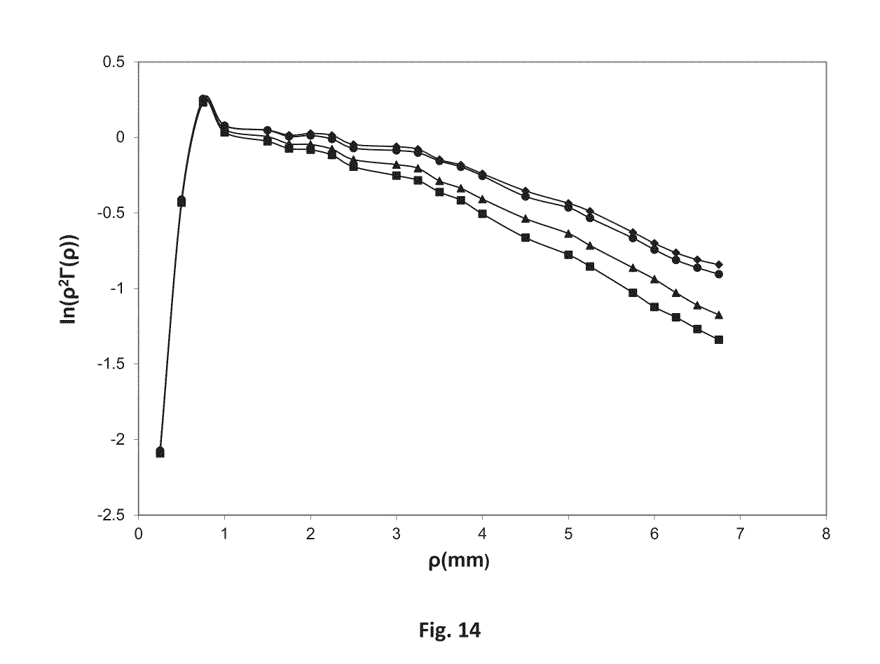

FIG. 15 presents the square slopes of ln(ρ²Γ(ρ)) curves versus the varying absorption coefficients μₐ of all simulated tissues, as well as the predicted square slopes (according to the absorption and scattering coefficients that were inserted into the simulation parameters) calculated from Eq. 2. According to Eq. 2 and 3, the square slope of the resultant linear curve is equal to 3·μ′ₛ. The simulated linear relation resulting from FIG. 15 was: (slope²)=4.75μₐ−0:027. The μ′ₛ that was inserted into the simulation parameters was 1.6 mm⁻¹, resulting in the theoretical product 3·μ′ₛ of 4.8. The resulted slope in the above equation, pointing on 4.75, is almost identical to the theoretical product. Thus, these simulation results suggest that the term ln(ρ²Γ(ρ)) is adequate for the correlation between DR measurements and the optical properties of low absorbing media.

### DR Measurements of Solid Phantoms:

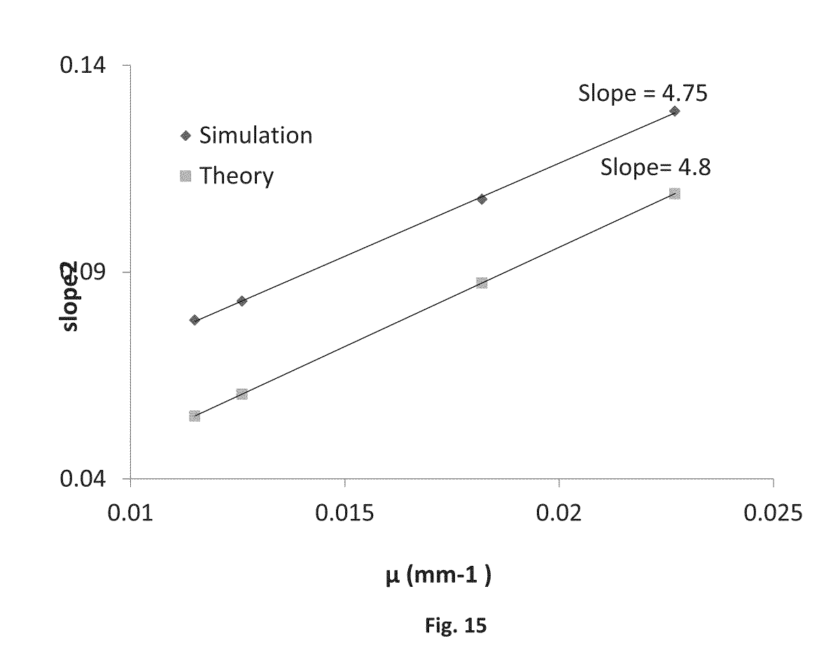

The reflected light intensity from eight different solid phantoms was measured using the experimental set-up described above. Representative results of the reflected light intensity profiles, plotted as the logarithm of the product between the square distance and the reflectance versus the distance, are presented in FIG. 16. The experimental results well confirm the analytical predictions of the diffusion theory: the larger pa, the sharper the graph's slope. The phantoms' absorption coefficients were: μₐ=0.0115 mm⁻¹ (marked by “⋄”); 0.0126 mm⁻¹ (marked by circles); 0.018 mm⁻¹ (marked by triangles); and 0.0227 mm⁻¹ (marked by squares) and their ln(ρ²Γ(ρ)) slopes present increasing respective values (in absolute units) of: 0.003±0.0009, 0.01±0.0005, 0.025±0.0001 and 0.06±0.0002, respectively.

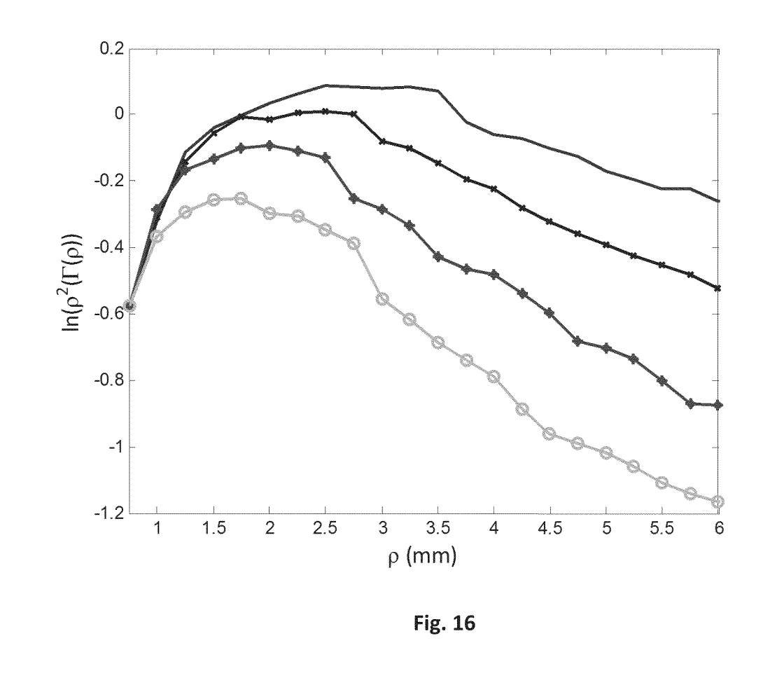

The slopes were calculated from the distance of mm, where the graphs start a monotonous decay. Some deviations in the slopes values occurred, as presented in FIG. 17, mainly since the resultant phantoms were not totally homogeneous and therefore some variations in their optical properties occurred.

FIG. 17 presents the increasing values of the ln(ρ²Γ(ρ)) square slopes versus the absorption coefficients of the phantoms. The slope of ln(ρ²Γ(ρ)) one gets a simple correlation to the tissue-like phantoms' optical properties. Since the varying component in the phantoms' solutions was the ink (the absorbing component), the square slope of each graph is equal to 3·μ′ₛ. The resulting equation for the correlation between the square slopes of ln(ρ²Γ(ρ)) and the absorption coefficients of the irradiated phantoms was: (slope)=4.35μₐ−0.038, referred to as Eq. (8).

According to the analytical prediction and our simulation results, the resulted slope of the linear curve, which is equal to 4.35, represents the product 3·μ′ₛ. The resultant μ′ₛ is 1.45 mm⁻¹ This is similar to the resultant μ′ₛ of phantom containing 2% IL, as was suggested by Cubeddu et al., 1997. Using Eq. (8), the absorption coefficient of any phantom, presenting μ′ₛ=1.45 mm⁻¹, can be deduced from the slope of its reflected light intensity plotted as ln(ρ²Γ(ρ)).

### DR Measurements of Solid Phantoms Containing GNR:

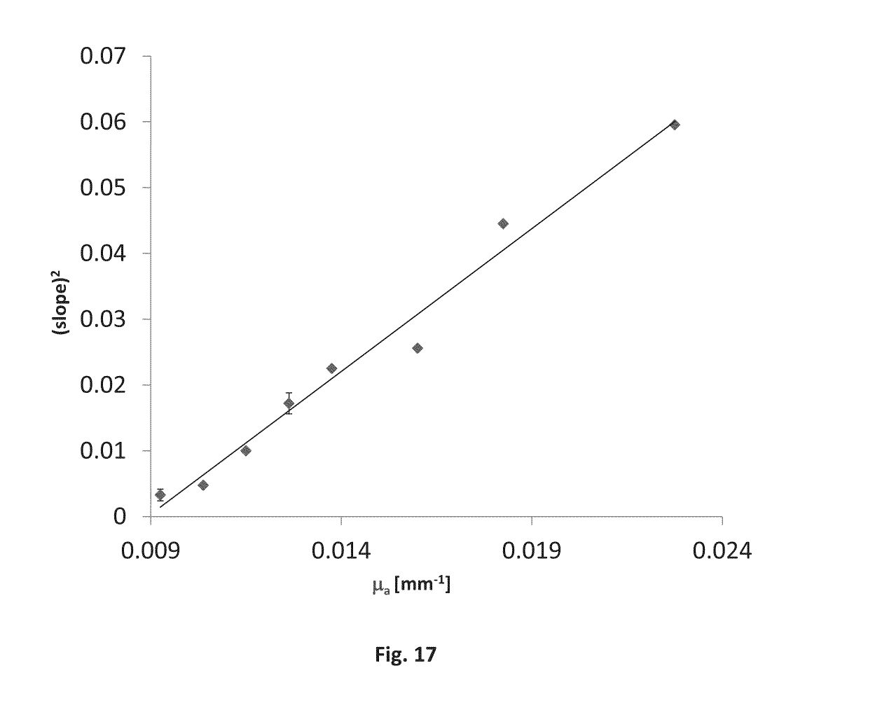

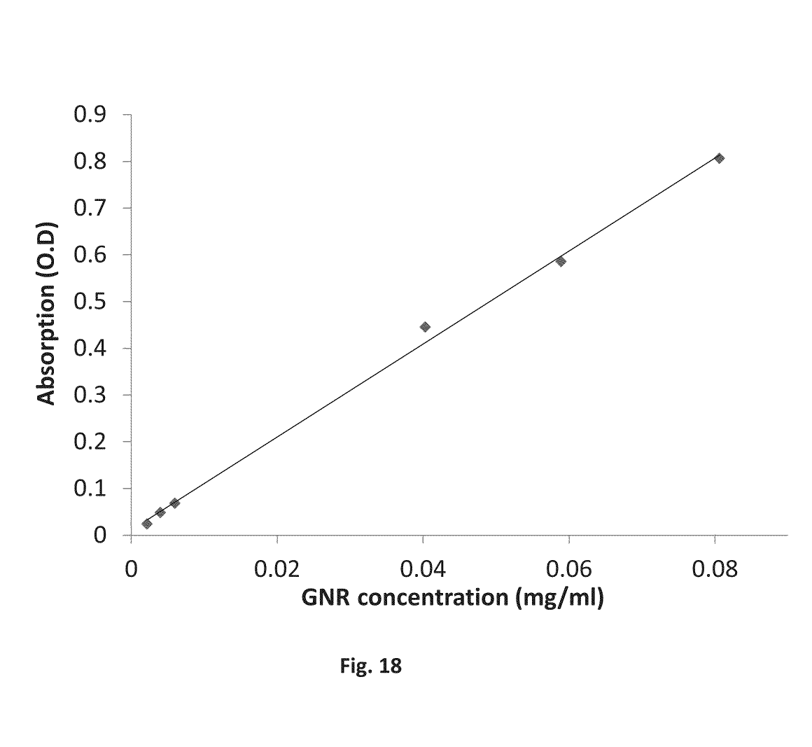

FIG. 18 presents the absorption spectrophotometer results of GNR solutions (the GNR were suspended in double distilled water) with the varying GNR concentrations of 0.002, 0.004, 0.006, 0.04, 0.06, 0.08 mg/ml. From the graph's slope we found that the mean extinction coefficient of the GNR was approximately 1 ml/(mm·mg). Combining DR measurements of solid phantoms with the GNR optical properties, DR measurements of the six phantoms presented in Table 2 were performed. By multiplying this extinction coefficient with the known GNR concentration, the GNR absorption coefficients in each phantom were calculated. FIG. 19 shows representative results for the DR measurements (plotted as ln(ρ²Γ(ρ)) of three solid phantoms containing 0.002, 0.003 and 0.006 mg/ml of GNR. The results indicate that the higher the GNR concentration, the sharper the reflectance graph's slope. The six phantoms' square slopes of the ln(ρ²Γ(ρ)) profiles are presented in the following Table 4:

TABLE 4

| Calculated μₐ [mm⁻¹] | (slope)² | Experimental μₐ [mm⁻¹] |
|---|---|---|
| 0.0123 | 0.011 ± 0.006 | 0.012 ± 0.0007 |
| 0.0132 | 0.0143 ± 0.003 | 0.0131 ± 0.0031 |
| 0.0152 | 0.0256 ± 0.003 | 0.0146 ± 0.0007 |
| 0.0216 | 0.0625 ± 0.004 | 0.0229 ± 0.001 |
| 0.0312 | 0.0137 ± 0.006 | 0.0397 ± 0.0004 |
| 0.0986 | 0.042 ± 0.001 | 0.104 ± 0.0002 |

As mentioned in the Material and Method section, the GNR have high absorption at 650 nm but negligible scattering properties in this wavelength. Therefore, the increase in each of the graphs' slopes is due to the increase in the absorption of the irradiated phantom, resulting from the presence of the GNR (since the ink concentration in each phantom has been kept constant).

The absorption coefficients of the phantoms were calculated according to the sum of the GNR and ink absorption coefficient. The resultant total μₐ of each of the six measured phantoms are presented in the left column of Table 3. The middle column of the table presents the square slopes of ln(ρ²Γ(ρ)) from which the absorption coefficients of each phantom were calculated. The right column in Table 3 presents the resultant absorption coefficients of the irradiated phantoms containing the GNR, as were calculated from the square slopes using on Eq. (8). One can notice the good correlation between the calculated and the experimental absorption coefficient values. These results confirm that the DR measurements can serve for the absorption coefficient extraction of turbid media containing GNR.

### In Vivo Results:

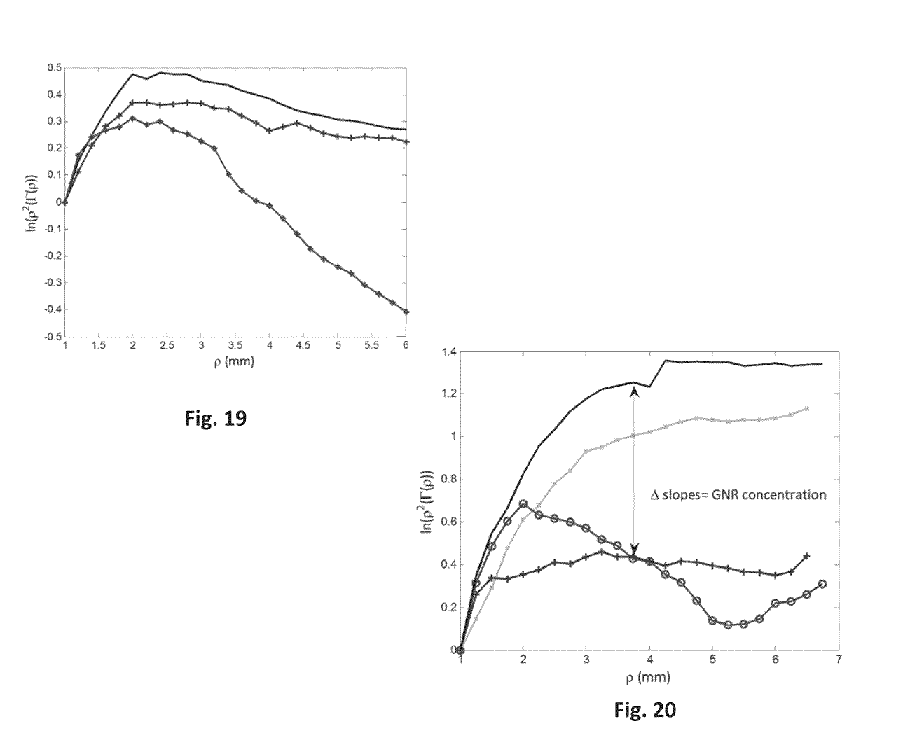

Tumor-bearing mice were irradiated and the reflected light intensity was collected using the optical set-up described in the Materials and Methods section above. The reflectance measurements of the tumor were performed before the GNR injection and for several delay times post-injection (15 minutes, 5 and 10 hours). FIG. 20 shows representative reflected light intensity profiles as was collected from three mice. Before the GNR injection, no negative slope was observed, pointing on low absorption and scattering properties of the tissue. The slope became sharper after 15 minutes but still not sharp enough for the GNR absorption coefficient calculation. After 5 and 10 hours, the slope of ln(ρ²Γ(ρ)) was sharp enough for this calculation.

Since before the GNR injection the graph's slope did not present any decay, the slope after 5 and 10 hours directly indicates the GNR accumulation in tumor. As a result, the GNR absorption coefficient was deduced from the change in the graphs' slopes (Δslope) for the different delay times, compared to the slope of the tumor's DR profile before the GNR injection.

Table 5 below shows the Δslopes of the reflected light intensity presented in FIG. 20:

TABLE 5

| | | Experiment |
|---|---|---|
| Time | Δslope² | al μₐ [mm⁻¹] |
| Before GNR injection | 0 | 0 |
| 15 min post GNR injection | — | — |
| 5 h post GNR injection | 0.0036 | 0.0096 |
| 10 h post GNR injection | 0.0275 | 0.015 |

The GNR absorption coefficients in the different delay times were first deduced by comparing the measured Δslopes with the slopes of the DR curves of the phantoms containing GNR (presented in Table 4).

The measured square Δslope 10 hours post injection was (Δslope)²=0.0275, similar to the square slope of the phantom presenting μₐ=0.015 mm⁻¹ As was shown in Table 3, this absorption coefficient also resulted from Eq. (8), which was extracted from the DR measurements of phantoms with a specific scattering (due to the constant concentration of 2% IL). Despite the unknown scattering properties of the mice tumor, the scattering properties of the phantoms were adjusted to skin scattering properties (Dam et al., 2001).

The mice tumors (without GNR) presented a slope similar to the slope of a ‘regular’ tissue (without tumor, data not shown) therefore, the tumor optical properties can be related to those of the ‘regular’ tissue. Thus, Eq. (8) can also fit for the calculation of GNR concentration in the tumor.

Accordingly, the absorption coefficient of the GNR in tumor 5 hours post injection was also calculated from this equation and the result was 0.0096 mm⁻¹ Using the extinction coefficient of GNR (approximately 1 ml/(mm·mg), see FIG. 18), the GNR concentration in the tumor 5 and 10 hours post GNR injection was 0.0096 mg/ml and 0.015 mg/ml, respectively. As a control measurement, the total concentration of GNR within the tumor was determined using the FAA spectroscopy and the result was 0.0218 mg/ml. Our GNR concentration result 10 hours post injection, calculated using Eq. (8), presents a lower value than the FAA result. This is obvious since the FAA measured the concentration of the GNR present in the entire tumor, on its surface and within the tumor (which was considered as a sphere, with a diameter of approximately 9 mm), while the reflectance measurements presented a maximal length of 6.25 mm and an approximated penetration depth of 9 mm. The DR measurements detected only part of the GNR concentration in tumor. Still, the slope after 10 hours reflects a high percentage (68%) of the total GNR concentration, indicating an inhomogeneous dispersing of the GNR in the tumor.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments and/or by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a sub-combination or variation of a sub-combination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what essentially incorporates the essential idea of the invention.

Although the invention has been described in detail, nevertheless changes and modifications, which do not depart from the teachings of the present invention, will be evident to those skilled in the art. Such changes and modifications are deemed to come within the purview of the present invention and the appended claims.

### REFERENCES

Ankri R, V. Peretz, M. Motiei et al., “A new method for cancer detection based on diffusion reflection measurements of targeted gold nanorods” Int. J. Nanomedicine, 7, 449-455, 2012 a
Ankri R, H. Duadi, M. Motiei, D. Fixler, “In-vivo tumor detection using diffusion reflection measurements of targeted gold nanorods—a quantitative study,” J. Biophotonics, 5(3), 263-273, 2012b.
Ankri R, A. Meiri, S L Lau, M. Motiei, Rachela Popovtzer, D. Fixler, “Intercoupling surface plasmon resonance and diffusion reflection measurements for real-time cancer detection” J. Biophotonics, 6(2), 188-196, 2013 (published online 28 Mar. 2012).
Baselga J. The EGFR as a target for anticancer therapy—focus on cetuximab. Eur J Cancer, 37:16-22, 2001
Bigio I J, Bown S G, Briggs G, et al. Diagnosis of breast cancer using elastic-scattering spectroscopy: preliminary clinical results. J Biomed Opt. 5:221-228, 2000.
Bonner R F, R. Nossal, and. Weiss G H. Photon Migration in Tissues, B. Chance (ed.) (Plenum, New-York, 1998), pp. 11-23.
Cerussi A E, Tanamai V W, Hsiang D, Bulter J, Mehta R. S, and Tromberg B J, Phil. Trans. R Soc. A 369, 4512-4530 (2011).
Cubeddu R, Pifferi A, Taroni P, Torricelli A, Valentini G. A solid tissue phantom for photon migration studies. Phys Med Biol. 42: 1971-1979, 1997.
Dagdug L, Weiss G H, and Gandjbakhche A H. Phys. Med. Biol. 48, 1361-1370 (2003).
Dam J S, Pedersen C B. Dalgaard T, Fabricius P E, Aruna P, Andersson-Engels S, Fiber-optic probe for noninvasive real-time determination of tissue optical properties at multiple wavelengths. Appl Opt. 40:1155-1164, 2001.
Draine B T, and Flatau P J. User Guide to the Discrete Dipole Approximation Code DDSCAT 7.0. http://arXivorg/abs/10021505v1 809, 1-79 (2004).
Feather J W, Ellis D J, and Leslie G. Phys. Med. Biol. 33, 711-722 (1988).
Gobin A M, Lee M H, Halas N J, James W D, Drezek R A, West J L. Near-infrared resonant nanoshells for combined optical imaging and photothermal cancer therapy. Nano Lett. 7: 1929-1934, 2007.
Hielscher A H, Mourant J R, and Bigio U. Appl. Opt. 36, 125-135 (1997).
Huang X, El-Sayed I H, Qian W, El-Sayed A M. Cancer cell imaging and photothermal therapy in the near-infrared region by using gold nanorods. J Am Chem Soc. 128:2115-2120, 2006.
Jacques S T, Pogue B W. Tutorial on diffuse light transport. J Biomed Opt. 13:0413021-0413019, 2008.
Lal S, Clare S E, Halas N J. Nanoshell-enabled photothermal cancer therapy: impending clinical impact. Acc Chem Res. 4:1842-1851, 2008.
Lin A J, Koike M A, Green K N. et al. “Spatial frequency domain imaging of intrinsic optical property contrast in a mouse model of alzheimer's disease,” Ann. Biomed. Eng., 39, 1349-1357 (2011).
Lowery A R, Gobin A M, Day E S, Halas N J, West J L Immunonanoshells for targeted photothermal ablation of tumor cells. Int J Nanomedicine 1:149-154, 2006.
Mallidi S, Larson T, Tam J, Joshi P P, Karpiouk A, Sokolov K, and Emelianov S. Nano Letters 9, 2825-2831 (2009).
Nikoobakht B, El-Sayed M A. Preparation and growth mechanism of gold nanorods (NRs) using seed-mediated growth method. Chem Mater. 2003; 15: 1957-1962
Pham T H, Coquoz O, Fishkin J B. et al. “Broad bandwidth frequency domain instrument for quantitative tissue optical spectroscopy,” Review of scientific instruments, 71, (2000).
Reuveni T, Motiei M, Romman Z, Popovtzer A, Popovtzer R. Targeted gold nanoparticles enable molecular C T imaging of cancer: an in vivo study. Int J Nanomedicine 6:2859-2864, 2011.
Schmitt J M, Zhou G X, Walker E C, Wall R T. Multi-layer model of photon diffusion in skin. J Opt Soc Am A. 7:2141-2153, 1990.
Shimada M, Sato C, Hoshi Y, Yamada Y. Estimation of the absorption coefficients of two-layered media by a simple method using spatially and time-resolved reflectance. Phys. Med. Biol. 54:5057-5071, 2009.
Stanton P, Richards S, Reeves J, et al. Epidermal growth factor receptor expression by human squamous cell carcinomas of the head and neck, cell lines and xenografts. Br J Cancer 70:427-433, 1994.
Tromberg B J, Coquoz O, Fishkin J B, et al. “Non-invasive measurements of breast tissue optical properties using frequency-domain photon migration,” Phil. Trans. R. Soc. Lond. B 352, 661-668 (1997).
Yang Y, Celmer E J, Koutcher J A, and Alfano R R. J. Clin. Laser. Med. Surg. 19, 35-39 (2001).
Zaccanti G, Alianelli L, Blumetti C, and Carraresi S. Opt. Lett. 24, 1290-1292 (1999).
Zhu G, Palmer M, Breslin™, Harter J, and Ramanujam N. Lasers in Surgery and Medicine 38, 714-724 (2006).

1. A non-invasive and real-time optical method for detection of cancerous cells, said method comprising the steps of:
a) optically irradiating with a light source outputting an optical signal of at least one wavelength, an area of a tissue in which targeted nanoparticles are accumulated;
b) identifying cancerous cells by measuring diffusion reflection of said area of the irradiated tissue where the cancerous cells and the nanoparticles are located; and
c) outputting data indicative of the identified cancerous cells.

2. The method according to claim 1, wherein said identifying step comprises:
(i) detecting diffusion reflection intensities of the area of the irradiated tissue for different distances between said light source and a detector; and
(ii) calculating optical properties of the irradiated tissue based on the detected reflected intensity behavior in relation to said distances using a diffusion reflection based mathematical model.

3. The method according to claim 2, wherein said optical properties comprise absorption and/or scattering properties of the irradiated tissue.

4. The method according to claim 1, wherein said irradiation is carried out with a laser device alone or together with at least one optical fiber for guiding light outputted from the laser device to the cancerous cells area.

5. The method according to claim 1, wherein said at least one wavelength is in the range of 650-900 nm.

6. The method according to claim 1, wherein said cancerous cells is of a superficial tumor.

7. The method according to claim 6, wherein said superficial tumor is head and neck cancer or melanoma.

8. The method according to claim 1, wherein the nanoparticles are gold nanorods.

9. The method according to claim 8, wherein the gold nanorods are conjugated with targeting moieties specific to receptors of the cancerous cells and said conjugated gold nanorods are administered to a patient before the optical irradiation of the cancerous tissue, wherein said targeting moieties are antibodies and the gold nanorods are coated with polyethyleneglycol.

10. The method according to claim 2, further comprising detecting wavelengths of light irradiated from the tissue and identifying concentration of cancerous cells in the irradiated tissue based on red-shift of the irradiated light caused by surface plasmon resonance of concentrated nanoparticles, wherein said tissue is irradiated by outputting an optical signal of multiple wavelengths for enhancing identification of cancerous cells concentration.

11. A system for non-invasive and real time optical detection of cancerous cells in an area of a tissue in which targeted nanoparticles are accumulated, said system comprising:
a) an optical source setup for irradiating said tissue, said optical source comprising a laser device configured for outputting an optical signal of at least one wavelength;
b) at least one detector configured for detecting light reflected from the irradiated tissue; and
c) a processing unit for receiving output of said at least one detector and identifying cancerous tissue by calculating optical properties of the irradiated tissue from the detected light, using a diffusion reflection based mathematical model.

12. The system according to claim 11, wherein said optical source setup further comprises at least one optical fiber for guiding light outputted by the laser device to the cancerous cells area, said laser device being configured for outputting an optical signal of a single wavelength or multiple wavelengths.

13. The system according to claim 12, wherein said optical source setup further comprises at least one micrometer plate attached to a distal edge of said at least one optical fiber for allowing changing the relative source-detector separation between the location of the optical fiber output and said at least one detector for measuring the diffusion reflection in the specific body area.

14. The system according to claim 11, wherein said system further comprises a signal collecting unit for collecting output signals from said at least one detector and outputting signal related data, said signal collecting unit is configured to transmit the signal related data to said processing system, and said signal collecting unit is an oscilloscope, a central processing unit (CPU) communicating with said processing unit or a software program operable through said processing unit capable of receiving input data from said at least one detector through hardware of said processing unit.

15. The system according to claim 11, wherein said optical source setup and/or said at least one detector is configured for changing its location for measuring irradiated light from various source-detector separations.

16. The system according to claim 15, wherein said detector is configured for being moved at predefined distance intervals for changing its relative location or said optical source setup is configured for being moved at predefined intervals for changing the relative location of an output of the light source.

17. The system according to claim 11, wherein said detector and/or said optical source setup is configured to allow continuous measuring of spatial reflectance from the irradiating tissue.

18. The system according to claim 11, wherein said optical source setup comprises at least one laser diode, each outputting an optical signal at a different narrow wavelength.

19. The system according to claim 11, wherein said at least one detector is further configured for detecting wavelength or frequency of the optical signal irradiated from said tissue and said processing unit is configured for identifying concentration of cancerous cells in the irradiated tissue based on intensity decay of the optical signal, caused by concentrated targeted nanoparticles.

20. The system according to claim 11, wherein said optical properties comprise absorption and/or scattering properties of the irradiated tissue.
